(19) United States
(12) Patent Application Publication
(10) Pub. No.: US 2022/0025424 A1
(43) Pub. Date: Jan. 27, 2022

(54) COMPOSITIONS AND METHODS FOR PRODUCING STEREOISOMERICALLY PURE AMINOCYCLOPROPANES

(71) Applicant: Imago Biosciences, Inc., San Carlos, CA (US)

(72) Inventors: Amy E. TAPPER, Boston, MA (US); Cassandra CELATKA, Hull, MA (US); Arthur Glenn ROMERO, Chesterfield, MO (US); John M. MCCALL, Boca Grande, FL (US); Toni CHANCELLOR, San Carlos, CA (US); He ZHAO, Madison, CT (US); Betina BIOLATTO, Manalapan, NJ (US); Jian-Xie CHEN, Schenectady, NY (US); Elisabeth C.A. BROT, Albany, NY (US); Peter C. MICHELS, Voorheesville, NY (US); Venkat K. CHARI, Schenectady, NY (US); Ian C. COTTERILL, Altamont, NY (US)

(21) Appl. No.: 16/326,498

(22) PCT Filed: Aug. 16, 2017
(86) PCT No.: PCT/US17/47192

§ 371 Date: Feb. 19, 2019

Related U.S. Application Data
(60) Provisional application No. 62/375,719, filed on Aug. 16, 2016.

(51) Int. Cl. C12P 41/00; C12P 13/00; C12N 9/04

(52) U.S. Cl. CPC C12P 41/002; C12Y 101/01; C12N 9/0006; C12P 13/005

(57) ABSTRACT

The present disclosure relates to compositions and methods for producing stereoisomerically pure aminocyclopropanes.

This application claims the benefit of U.S. Provisional Application No. 62/375,719, filed Aug. 16, 2016, the entirety of which is hereby incorporated by reference as if written herein in its entirety.

The present disclosure relates to compositions and methods for producing stereoisomerically pure aminocyclopropanes, more specifically to methods of using engineered ketoreductase enzymes to synthesize aminocyclopropanes.

Stereoisomerically pure substituted aminocyclopropanes are key chiral intermediates for the synthesis of KDM1A inhibiting compounds useful for treating hematologic disease such as sickle cell disease, thalassemia major, and other hemoglobinopathies as well as neoplasms and clonal disorders such as breast and prostate cancer, acute myelogenous leukemia, myeloproliferative neoplasia and myelodysplastic syndrome.

While various methods for producing these chiral intermediates are known, these methods suffer significant drawbacks, making them less than ideal for commercial scale synthesis. These drawbacks include multiple column chromatography separations, extra reaction steps, low yields, high reagent costs, less efficient (used only half of diastereomer intermediates), large volume of solvents, and extremely drying intermediates and solvents, making the process difficult to scale up. Given the importance of these key chiral intermediates in the synthesis of KDM1A inhibitors, compositions and methods useful for synthesizing these compounds in a cost effective and efficient manner would be highly desirable.

Thus, there remains a need for improved methods and compositions for synthesizing stereoisomerically pure aminocyclopropanes, more specifically to methods of using engineered ketoreductase enzymes to synthesize substituted aminocyclopropanes.

Accordingly, disclosed herein are compositions and methods for synthesizing stereoisomerically pure aminocyclopropanes. Advantages of the compositions and methods include, in certain embodiments, one or more of: 1) no column chromatography purification; 2) simple reaction operation; 3) no extremely anhydrous intermediates and solvents; 4) simple work-ups; 5) stereogenic center introduced by bio transformation; and 6) high overall yield.

In certain embodiments, the methods use engineered ketoreductase enzymes to synthesize substituted aminocyclopropanes.

Accordingly, provided is a composition comprising:
a) a compound of Formula II:

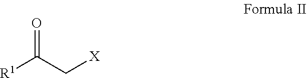

or a salt thereof; wherein:
   X is chosen from Cl, Br, and I;
   R¹ is chosen from aryl and heteroaryl, any of which is optionally substituted with between 1 and 3 R³ groups;
   each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵;
   R⁴ and R⁵ are independently chosen from hydrogen, and lower alkyl;
   or R⁴ and R⁵ may be taken together to form a nitrogen-containing heterocycloalkyl or heteroaryl ring, which is optionally substituted with lower alkyl; and
b) an engineered or isolated ketoreductase enzyme capable of stereoselectively reducing the oxo of Formula II to a hydroxyl group.

Also provided is a process for preparing a chiral halohydrin compound of Formula III:

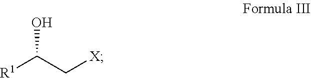

or a salt thereof; wherein:

X is chosen from Cl, Br, and I;

R¹ is chosen from aryl and heteroaryl, any of which is optionally substituted with between 1 and 3 R³ groups;

each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵;

R⁴ and R⁵ are independently chosen from hydrogen, and lower alkyl; or R⁴ and R⁵ may be taken together to form a nitrogen-containing heterocycloalkyl or heteroaryl ring, which is optionally substituted with lower alkyl; comprising the step of:
a) enantioselectively reducing a compound of Formula II:

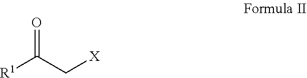

or a salt thereof, with an engineered or isolated ketoreductase enzyme capable of stereoselectively reducing the oxo to a hydroxyl group to provide the chiral halohydrin compound of Formula III:

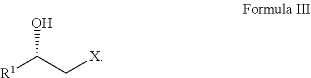

Provided is a process for preparing a chiral Cyclopropyl compound of Formula I

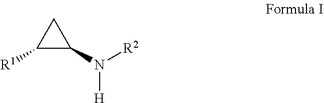

or a salt thereof; wherein:

R¹ is chosen from aryl and heteroaryl, any of which is optionally substituted with between 1 and 3 R³ groups;

R² is chosen from hydrogen and C(O)OR³;

each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵;

each R⁴ and R⁵ are independently chosen from hydrogen, and lower alkyl;

or R⁴ and R⁵ may be taken together to form a nitrogen-containing heterocycloalkyl or heteroaryl ring, which is optionally substituted with lower alkyl; comprising the steps of:
a) enantioselectively reducing a compound of Formula II:

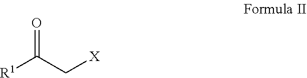

or a salt thereof; with an engineered or isolated ketoreductase enzyme capable of stereoselectively reducing the oxo to a hydroxyl group to provide a chiral halohydrin compound of Formula III:

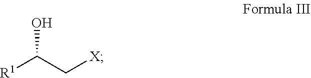

wherein X is chosen from Cl, Br, and I,
b) treating the compound of Formula III with a base to provide the epoxide of Formula IV or a salt thereof:

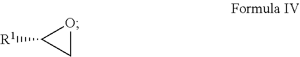

c) treating the compound of Formula IV with a Wadsworth-Emmons reagent and a base to provide the cyclopropyl ester of Formula V or a salt thereof:

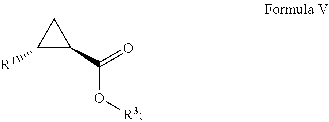

d) treating the compound of Formula V with a reagent to provide the cyclopropyl acid of Formula VI or a salt thereof:

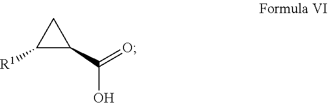

e) treating the compound of Formula VI with azidization reagent, a base, and a alcohol of Formula VII:

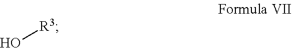

to provide the cyclopropyl carbamate of Formula VIII or a salt thereof:

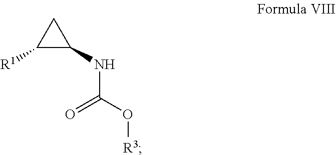

and, optionally,
f) treating the cyclopropyl carbamate of Formula VIII with a suitable deprotecting base or acid to provide the cyclopropyl amine of Formula IX or a salt thereof:

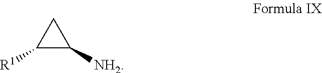

### BRIEF DESCRIPTION OF THE DRAWINGS

FIG. 1 shows the RP-HPLC chromatogram of the isolated Halohydrin lot #1; Panel A: Full chromatogram; Panel B: Expanded version of the chromatogram;

FIG. 2 shows the RP-HPLC chromatogram of the isolated Halohydrin lot #2; Panel A: Full chromatogram; Panel B: Expanded version of the chromatogram;

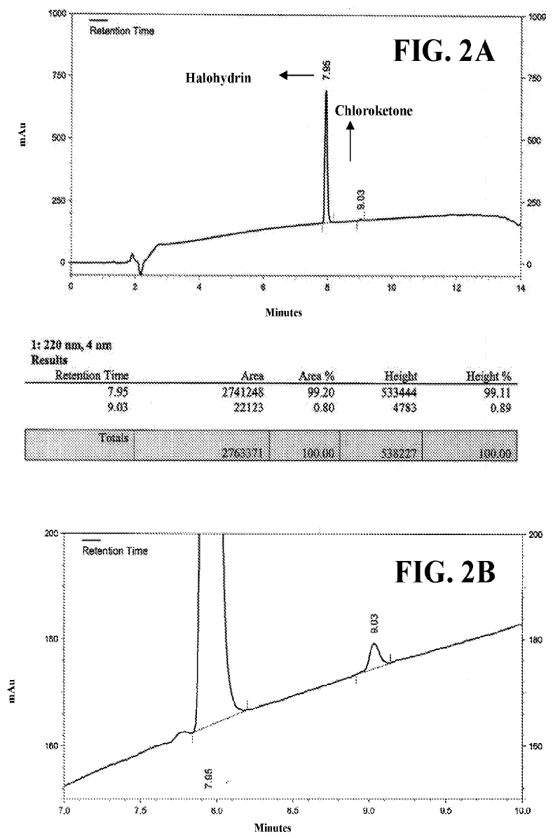

FIG. 3 shows the Chiral HPLC chromatogram of the isolated S-Halohydrin lot #1; Panel A: Full chromatogram; Panel B: Expanded version of the chromatogram

FIG. 4 shows the Chiral HPLC chromatogram of the isolated S-Halohydrin lot #2; Panel A: Full chromatogram; Panel B: Expanded version of the chromatogram;

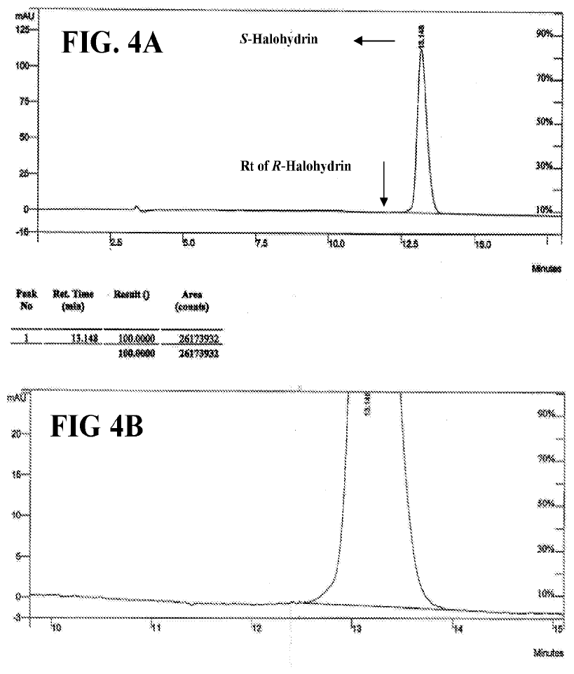

FIG. 5 shows the 1H NMR spectrum (CDCl3, 500 MHz) of Halohydrin lot #1; and

FIG. 6 shows the ¹H NMR spectrum (CDCl₃, 500 MHz) of Halohydrin lot #2.

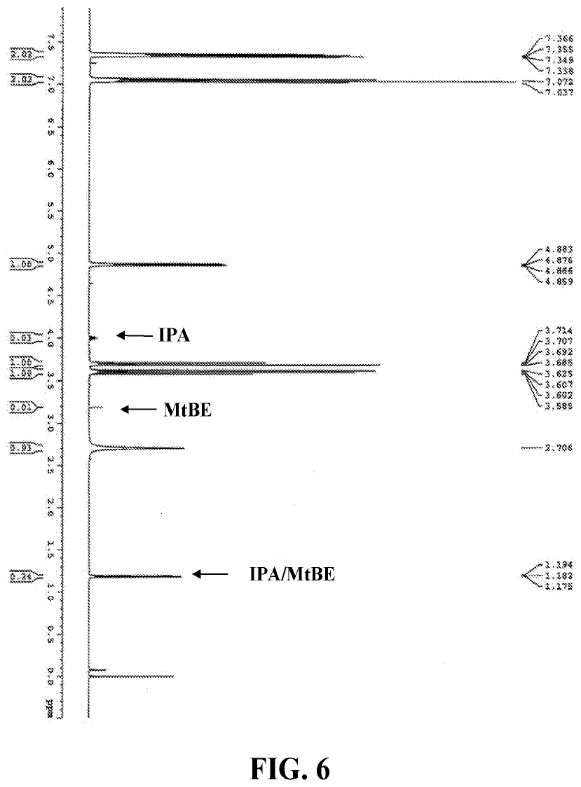

FIG. 7 shows the chiral HPLC analysis of halohydrin from KRED P1-F07 ketone reduction at 35° C.

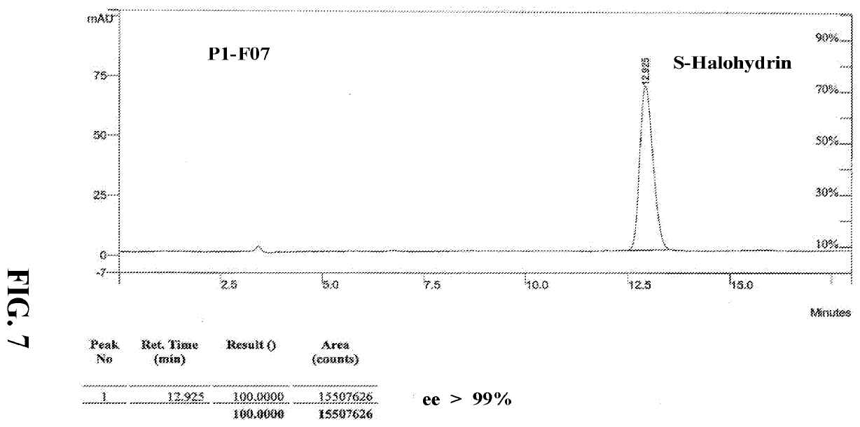

FIG. 8 shows the time course of KRED P2-G03 and KRED P1-F07 (0.5 g/L) reduction of 2-chloro-4′-fluoroacetophenone (150 g/L) to the k-halohydrin at 35° C.

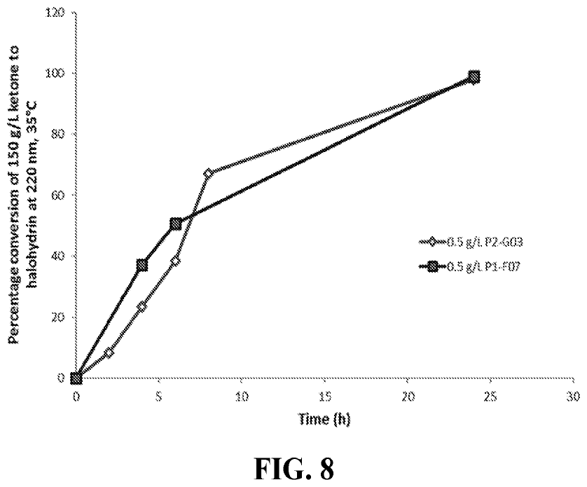

### DETAILED DESCRIPTION

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “alkylsulfonyl” as used herein, means an alkyl group, as defined herein, appended to the parent molecular moiety through a sulfonyl group, as defined herein.

Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups is optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched-chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms.

The term “alkynylene” refers to a carbon-carbon triple bond attached at two positions such as ethynylene (—C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R are independently chosen from hydrogen, alkyl, hydroxyalkyl, acyl, heteroalkyl, aryl, cycloalkyl, heteroaryl, and heterocycloalkyl, any of which may themselves be optionally substituted. Additionally, R and R′ may combine to form heterocycloalkyl, either of which is optionally substituted.

The term “amino acid”, as used herein, alone or in combination, refers to a —NHCHRC(O)O— group, which may be attached to the parent molecular moiety to give either an N-terminus or C-terminus amino acid, wherein R is independently chosen from hydrogen, alkyl, aryl, heteroaryl, heterocycloalkyl, aminoalkyl, amido, amidoalkyl, carboxyl, carboxylalkyl, guanidinealkyl, hydroxyl, thiol, and thioalkyl, any of which themselves is optionally substituted. The term C-terminus, as used herein, alone or in combination, refers to the parent molecular moiety being bound to the amino acid at the amino group, to give an amide as described herein, with the carboxyl group unbound, resulting in a terminal carboxyl group, or the corresponding carboxylate anion. The term N-terminus, as used herein, alone or in combination, refers to the parent molecular moiety being bound to the amino acid at the carboxyl group, to give an ester as described herein, with the amino group unbound resulting in a terminal secondary amine, or the corresponding ammonium cation. In other words, C-terminus refers to —NHCHRC(O)OH or to —NHCHRC(O)O⁻ and N-terminus refers to H₂NCHRC(O)O— or to H₃N⁺CHRC(O)O—.

The term “aryl”, as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄═ derived from benzene. Examples include benzothiophene and benzimidazole.

The term “biphenyl” as used herein refers to two phenyl groups connected at one carbon site on each ring.

The term “carbamate,” as used herein, alone or in combination, refers to an ester of carbamic acid (—NHCOO—) which may be attached to the parent molecular moiety from either the nitrogen or acid end, and which is optionally substituted as defined herein.

The term “O-carbamyl” as used herein, alone or in combination, refers to a —OC(O)NRR′ group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms.

Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halohydrin,” as used herein, alone or in combination, refers to a compound or functional group in which one carbon atom has a halogen substituent, and another carbon atom has a hydroxyl substituent, typically on adjacent carbons.

The term “guanidine”, as used herein, alone or in combination, refers to —NHC(═NH)NH₂, or the corresponding guanidinium cation.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogen atoms are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms chosen from O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom chosen from O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furanyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, azepinyl, diazepinyl, benzazepinyl, and the like. Exemplary tricyclic heterocyclic groups include carbazolyl, benzidolyl, phenanthrolinyl, dibenzofuranyl, acridinyl, phenanthridinyl, xanthenyl and the like.

The term “heteroarylalkyl” as used herein alone or as part of another group refers to alkyl groups as defined above having a heteroaryl substituent.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, imidazolidinyl, isoindolinyl, morpholinyl, oxazolidinyl, isoxazolidinyl, piperidinyl, piperazinyl, methylpiperazinyl, N-methylpiperazinyl, pyrrolidinyl, pyrazolidinyl, tetrahydrofuranyl, tetrahydropyridinyl, thiomorpholinyl, thiazolidinyl, diazepanyl, and the like. The heterocycle groups is optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “hydroxamic acid”, as used herein, alone or in combination, refers to —C(═O)NHOH, wherein the parent molecular moiety is attached to the hydroxamic acid group by means of the carbon atom.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which is optionally substituted as provided.

The term “lower heteroaryl,” as used herein, alone or in combination, means either 1) monocyclic heteroaryl comprising five or six ring members, of which between one and four said members may be heteroatoms chosen from O, S, and N, or 2) bicyclic heteroaryl, wherein each of the fused rings comprises five or six ring members, comprising between them one to four heteroatoms chosen from O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen, lower alkyl, and lower heteroalkyl, any of which is optionally substituted. Additionally, the R and R′ of a lower amino group may combine to form a five- or six-membered heterocycloalkyl, either of which is optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphonate,” as used herein, alone or in combination, refers to a —P(═O)(OR)₂ group, wherein R is chosen from alkyl and aryl. The term “phosphonic acid”, as used herein, alone or in combination, refers to a —P(═O)(OH)₂ group.

The term “phosphoramide”, as used herein, alone or in combination, refers to a —P(═O)(NR)₃ group, with R as defined herein.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethoxy” refers to a X₃CO— group where X is a halogen.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. Similarly, when a designation such as “n” which may be chosen from a group or range of integers is designated to be 0, then the group which it designates is either absent, if in a terminal position, or condenses to form a bond, if it falls between two other groups.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which is optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated according to the Cahn-Ingold-Prelog priority rules by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art.

Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Ketoreductase” and “KRED” are used interchangeably herein to refer to a polypeptide that is capable of enantioselectively reducing the 2-oxo group of a 1-halo-2-oxo derivative to yield the corresponding syn l-halo-2-hydroxy derivative (a halohydrin). The polypeptide typically utilizes the cofactor reduced nicotinamide adenine dinucleotide (NADH) or reduced nicotinamide adenine dinucleotide phosphate (NADPH) as the reducing agent. Ketoreductases as used herein include naturally occurring (wild type) ketoreductases as well as non-naturally occurring engineered polypeptides generated by human manipulation. Ketoreductases are commercially available (e.g., from Codexis, Inc.) and may be screened (e.g., via the Codex® KRED screening kit) for optimal properties. Preferred ketoreductases are those which 1) yield the greatest conversion of starting material to desired product, 2) do so at the highest rate, 3) yield the desired enantiomer (e.g., the (S) enantiomer), and/or 4) have better solvent and temperature tolerance. Ketoreductases are commercially available, e.g. from Codexis®. In certain embodiments, suitable ketoreductases are those suitable for the reduction of α-haloketones and/or acetophenones to the corresponding alcohols. Examples include the ketoreductases disclosed in, e.g., U.S. Pat. Nos. 7,879,585, 8,617,864, 8,796,002, 9,029,112, 9,296,992, 8,512,973, 8,748,143 B2, and U.S. Pat. No. 8,852,909. Codexis® ketoreductases include the ketoreductases identified as P1-A04, P1-B02, P1-B10, P1-B12, P1-C01, P1-H08, P1-H10, P2-B02, P2-C02, P2-C11, P2-D11, P1-F07 (P1F07/CDX023), P2-G03, and P2-H07.

“Coding sequence” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences.

The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BEAST and BEAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a “comparison window” to identify and compare local regions of sequence similarity.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly reported in the art (typically as a percentage) as the enantiomeric excess calculated therefrom according to the formula [major enantiomer-minor enantiomer]/[major enantiomer+minor enantiomer]. Where the stereoisomers are diastereomers, the stereoselectivity is referred to as diastereoselectivity, the fraction (typically reported as a percentage) of one diastereomer in the sum with others. In the context of the present disclosure, diastereoselectivity refers to the fraction (typically reported as a percentage) of the hydroxy oxo ester of structural formula (Ia) that gets converted into the syn dihydroxy ester of structural formula Ha, as opposed to the anti dihydroxy ester of formula lib. It may also be reported (typically as a percentage) as the diastereomeric excess calculated therefrom according to the formula [syn IIa-anti IIb]/[syn IIa+anti IIb].

### Compositions

The present disclosure provides compositions for synthesizing stereoisomerically pure aminocyclopropanes.

Provided is a composition comprising:
a) a compound of Formula II:

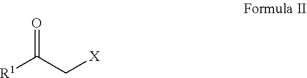

or a salt thereof; wherein:
   X is chosen from Cl, Br, and 1;
   R¹ is chosen from aryl and heteroaryl, any of which is optionally substituted with between 1 and 3 R³ groups;
   each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵;
   each R⁴ and R⁵ are independently chosen from hydrogen, and lower alkyl;
or R⁴ and R⁵ may be taken together to form a nitrogen-containing heterocycloalkyl or heteroaryl ring, which is optionally substituted with lower alkyl; and
b) an engineered or isolated ketoreductase enzyme capable of stereoselectively reducing the oxo of Formula II to a hydroxyl group.

In certain embodiments, R¹ is aryl, which is optionally substituted with between 1 and 3 R³ groups.

In certain embodiments, R¹ is phenyl, which is optionally substituted with between 1 and 3 R³ groups.

In certain embodiments, R¹ is heteroaryl.

In certain embodiments, R¹ is a 5-6 membered monocyclic or 8-12 membered bicyclic heteroaryl, in which between one and five ring members may be heteroatoms chosen from N, O, and S, and which is optionally substituted with between 1 and 3 R³ groups.

In certain embodiments, R¹ is a 5-6 membered monocyclic heteroaryl, in which between one and five ring members may be heteroatoms chosen from N, O, and S, and which is optionally substituted with 1 or 2 R³ groups.

In certain embodiments, R³ is halogen. In certain embodiments, R³ is fluorine.

In certain embodiments, R¹ is chosen from:

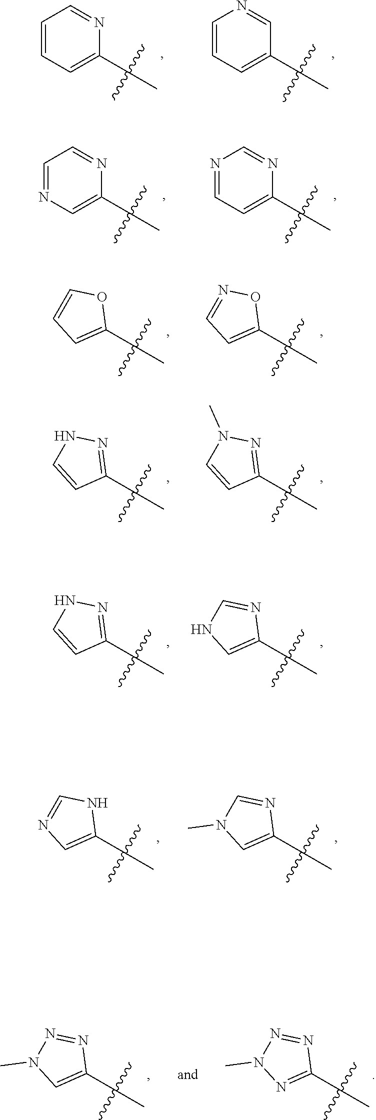

In certain embodiments, the ketoreductase enzyme yields a conversion of starting material to desired product of ≥95%; in certain embodiments, the ketoreductase enzyme yields a conversion of starting material to desired product of ≥97%; in certain embodiments, the ketoreductase enzyme yields a conversion of starting material to desired product of ≥98%; in certain embodiments, the ketoreductase enzyme yields a conversion of starting material to desired product of ≥99%. In any of the foregoing embodiments, the starting material may be 2-chloro-4′-fluoroacetophenone, and the desired product may be the (S)-halohydrin ((S)-2-Chloro-1-(4-fluorophenyl)ethanol), (S)-2-(4-Fluorophenyl)oxirane, or (1R,2S)-2-(4-fluorophenyl)cyclopropanamine hydrochloride.

In certain embodiments, the ketoreductase enzyme yields (S) enantiomeric excess of ≥95%; in certain embodiments, the ketoreductase enzyme yields (S) enantiomeric excess of ≥97%; in certain embodiments, the ketoreductase enzyme yields (S) enantiomeric excess of ≥98%; in certain embodiments, the ketoreductase enzyme yields (S) enantiomeric excess of ≥99%. In any of the foregoing embodiments, the (S) enantiomer may be the (S)-halohydrin.

In certain embodiments, the ketoreductase enzyme yields a high conversion rate of starting material to desired product. In certain embodiments, the ketoreductase enzyme has good temperature and solvent tolerance.

In certain embodiments, the ketoreductase is chosen from P1-A04, P1-B02, P1-B10, P1-B12, P1-C01, P1-H08, P1-H10, P2-B02, P2-C02, P2-C11, P2-D11, P1-F07, P2-G03, and P2-H07, which yielded ≥97% conversion of the acetophenone to the halohydrin. In certain embodiments, the ketoreductase is chosen from P1-A04, P1-B02, P1-B10, P1-B12, P1-H10, P2-C11, P1-F07, P2-G03, and P2-H07, which yielded ≥97% conversion of the acetophenone to the halohydrin and (S)-halohydrin enantiomeric excess of ≥97%. In certain embodiments, the ketoreductase is chosen from P1-A04, P1-B02, P1-B12, P1-H10, P2-C11, P1-F07, P2-G03, and P2-H07, which yielded ≥97% conversion of the acetophenone to the halohydrin and (S)-halohydrin enantiomeric excess of ≥98%. %. In certain embodiments, the ketoreductase is chosen from P1-A04, P1-B12, P1-H10, P1-F07, P2-G03, and P2-H07, which yielded ≥97% conversion of the acetophenone to the halohydrin and (S)-halohydrin enantiomeric excess of ≥99%. In certain embodiments, the ketoreductase is chosen from P1-F07 and P2-G03.

### Methods

The present disclosure provides methods for synthesizing stereoisomerically pure aminocyclopropanes.

Provided is a process for preparing a chiral halohydrin compound of Formula III:

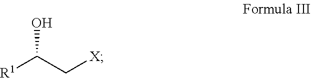

or a salt thereof; wherein:

X is chosen from Cl, Br, and I;

R¹ is chosen from aryl and heteroaryl, any of which is optionally substituted with between 1 and 3 R³ groups;

each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵;

each R⁴ and R⁵ are independently chosen from hydrogen, and lower alkyl; or R⁷ and R⁸ may be taken together to form a nitrogen-containing heterocycloalkyl or heteroaryl ring, which is optionally substituted with lower alkyl; comprising the step of:
a) enantioselectively reducing a compound of Formula II:

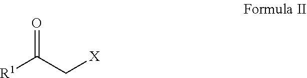

or a salt thereof; with an engineered or isolated ketoreductase enzyme capable of stereoselectively reducing the oxo to a hydroxyl group to provide the chiral halohydrin compound of Formula III:

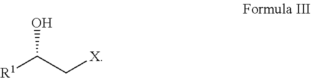

In certain embodiments, the process further comprises the step of:
b) recovering the chiral halohydrin compound of Formula III from the reaction mixture.

In certain embodiments, R¹ is aryl, which is optionally substituted with between 1 and 3 R³ groups.

In certain embodiments, R¹ is phenyl, which is optionally substituted with between 1 and 3 R³ groups.

In certain embodiments, R¹ is heteroaryl.

In certain embodiments, R¹ is a 5-6 membered monocyclic or 8-12 membered bicyclic heteroaryl, in which between one and five ring members may be heteroatoms chosen from N, O, and S, and which is optionally substituted with between 1 and 3 R³ groups.

In certain embodiments, R¹ is a 5-6 membered monocyclic heteroaryl, in which between one and five ring members may be heteroatoms chosen from N, O, and S, and which is optionally substituted with 1 or 2 R³ groups.

In certain embodiments, R is chosen from:

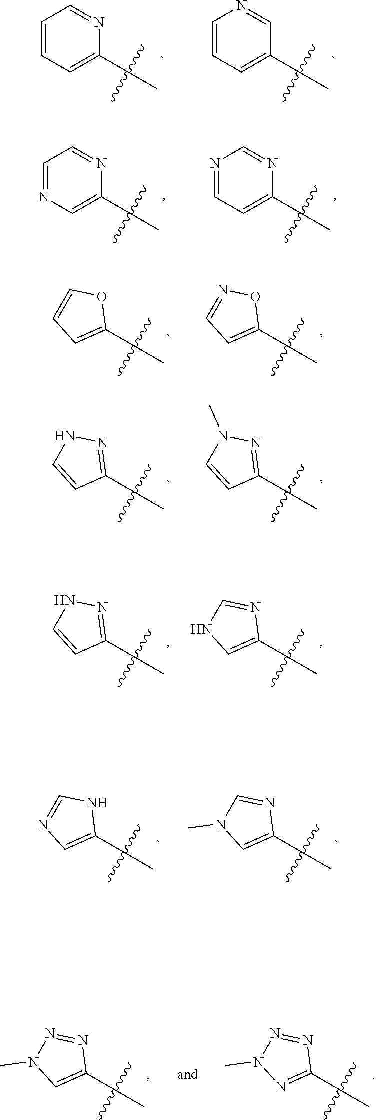

In certain embodiments, X is chloro.

In certain embodiments, the ketoreductase enzyme yields a conversion of starting material to desired product of ≥95%; in certain embodiments, the ketoreductase enzyme yields a conversion of starting material to desired product of ≥97%; in certain embodiments, the ketoreductase enzyme yields a conversion of starting material to desired product of ≥98%; in certain embodiments, the ketoreductase enzyme yields a conversion of starting material to desired product of ≥99%. In any of the foregoing embodiments, the starting material may be 2-chloro-4′-fluoroacetophenone, and the desired product may be the (S)-halohydrin ((S)-2-Chloro-1-(4-fluorophenyl)ethanol), (S)-2-(4-Fluorophenyl)oxirane, or (1R,2S)-2-(4-fluorophenyl)cyclopropanamine hydrochloride.

In certain embodiments, the ketoreductase enzyme yields (S) enantiomeric excess of ≥95%; in certain embodiments, the ketoreductase enzyme yields (S) enantiomeric excess of ≥97%; in certain embodiments, the ketoreductase enzyme yields (S) enantiomeric excess of ≥98%; in certain embodiments, the ketoreductase enzyme yields (S) enantiomeric excess of ≥99%. In any of the foregoing embodiments, the (S) enantiomer may be the (S)-halohydrin.

In certain embodiments, the ketoreductase enzyme yields a high conversion rate of starting material to desired product. In certain embodiments, the ketoreductase enzyme has good temperature and solvent tolerance.

In certain embodiments, the ketoreductase is chosen from P1-A04, P1-B02, P1-B10, P1-B12, P1-C01, P1-H08, P1-H10, P2-B02, P2-C02, P2-C11, P2-D11, P1-F07, P2-G03, and P2-H07, which yielded ≥97% conversion of the acetophenone to the halohydrin. In certain embodiments, the ketoreductase is chosen from P1-A04, P1-B02, P1-B10, P1-B12, P1-H10, P2-C11, P1-F07, P2-G03, and P2-H07, which yielded ≥97% conversion of the acetophenone to the halohydrin and (S)-halohydrin enantiomeric excess of ≥97%. In certain embodiments, the ketoreductase is chosen from P1-A04, P1-B02, P1-B12, P1-H10, P2-C11, P1-F07, P2-G03, and P2-H07, which yielded ≥97% conversion of the acetophenone to the halohydrin and (S)-halohydrin enantiomeric excess of ≥98%. %. In certain embodiments, the ketoreductase is chosen from P1-A04, P1-B12, P1-H10, P1-F07, P2-G03, and P2-H07, which yielded ≥97% conversion of the acetophenone to the halohydrin and (S)-halohydrin enantiomeric excess of ≥99%. In certain embodiments, the ketoreductase is chosen from P1-F07 and P2-G03.

In certain embodiments, the provided chiral halohydrin compound is substantially pure in the enantiomer of structural formula III. In certain embodiments, the provided chiral halohydrin compound is at least 99% pure in the enantiomer of structural formula III.

In certain embodiments, the process is carried out with whole cells that express the ketoreductase enzyme, or an extract or lysate of such cells.

In certain embodiments, the ketoreductase is isolated and/or purified.

In certain embodiments, the enantioselective reduction reaction is carried out in the presence of a cofactor for the ketoreductase and optionally a regeneration system for the cofactor.

In certain embodiments, the process is carried out at a temperature in the range of about 15° C. to about 75° C.

In certain embodiments, the process is carried out at a pH in the range of about pH 5 to pH 8.

In certain embodiments, the weight ratio of the oxo compound of structural formula II to the ketoreductase enzyme is in the range of about 10:1 to 200:1.

In certain embodiments, the process is carried out in the presence of a cofactor and optionally a cofactor regeneration system. In particular embodiments, the cofactor is NADH and/or NADPH, and in which the weight ratio of the cofactor to the ketoreductase enzyme is in the range of about 10:1 to 100:1. In particular embodiments, the cofactor regenerating system comprises glucose dehydrogenase and glucose; formate dehydrogenase and formate; or isopropanol and a secondary alcohol dehydrogenase.

Provided is a process for preparing a chiral cyclopropyl compound of Formula I

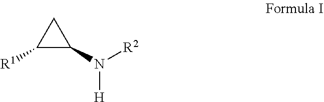

or a salt thereof; wherein:

R¹ is chosen from aryl and heteroaryl, any of which is optionally substituted with between 1 and 3 R³ groups;

R² is chosen from hydrogen and C(O)OR³;

each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵;

each R⁴ and R⁵ are independently chosen from hydrogen, and lower alkyl; or R⁴ and R⁵ may be taken together to form a nitrogen-containing heterocycloalkyl or heteroaryl ring, which is optionally substituted with lower alkyl; comprising the steps of:
a) enantioselectively reducing a compound of Formula II:

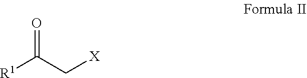

or a salt thereof; with an engineered or isolated ketoreductase enzyme capable of stereoselectively reducing the oxo to a hydroxyl group to provide a chiral halohydrin compound of Formula III:

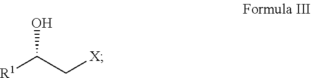

wherein X is chosen from Cl, Br, and I,
b) treating the compound of Formula III with a base to provide the epoxide of Formula IV or a salt thereof:

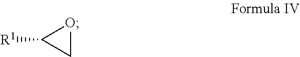

c) treating the compound of Formula IV with a Wadsworth-Emmons reagent and a base to provide the cyclopropyl ester of Formula V or a salt thereof:

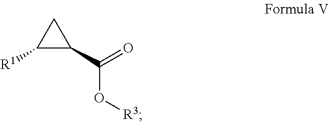

d) treating the compound of Formula V with a reagent to provide the cyclopropyl acid of Formula VI or a salt thereof:

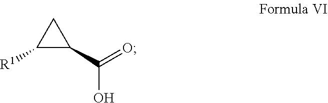

e) treating the compound of Formula VI with azidization reagent, a base, and a alcohol of Formula VII:

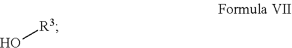

to provide the cyclopropyl carbamate of Formula VIII or a salt thereof:

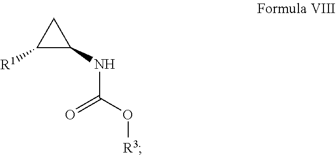

f) treating the cyclopropyl carbamate of Formula VIII with a suitable deprotecting base or acid to provide the cyclopropyl amine of Formula IX or a salt thereof:

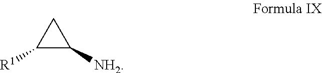

In certain embodiments, the process further comprises step f: treating the cyclopropyl carbamate of Formula VIII with a suitable deprotecting base or acid to provide the cyclopropyl amine of Formula IX or a salt thereof.

In certain embodiments, R¹ is aryl, which is optionally substituted with between 1 and 3 R³ groups.

In certain embodiments, R¹ is phenyl, which is optionally substituted with between 1 and 3 R³ groups.

In certain embodiments, R¹ is heteroaryl.

In certain embodiments, R¹ is a 5-6 membered monocyclic or 8-12 membered bicyclic heteroaryl, in which between one and five ring members may be heteroatoms chosen from N, O, and S, and which is optionally substituted with between 1 and 3 R³ groups.

In certain embodiments, R¹ is a 5-6 membered monocyclic heteroaryl, in which between one and five ring members may be heteroatoms chosen from N, O, and S, and which is optionally substituted with 1 or 2 R³ groups.

In certain embodiments, R¹ is chosen from:

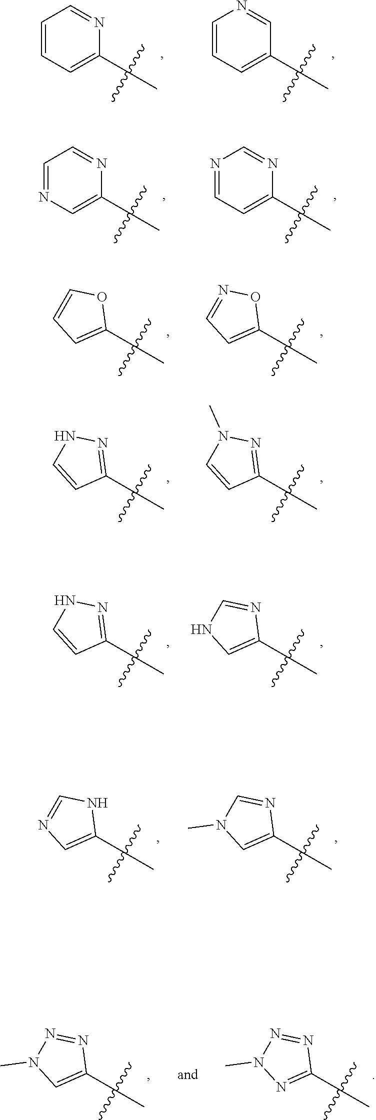

In certain embodiments, X is chloro.

In certain embodiments, the ketoreductase enzyme yields a conversion of starting material to desired product of ≥95%; in certain embodiments, the ketoreductase enzyme yields a conversion of starting material to desired product of ≥97%; in certain embodiments, the ketoreductase enzyme yields a conversion of starting material to desired product of ≥98%; in certain embodiments, the ketoreductase enzyme yields a conversion of starting material to desired product of ≥99%. In any of the foregoing embodiments, the starting material may be 2-chloro-4′-fluoroacetophenone, and the desired product may be the (S)-halohydrin ((S)-2-Chloro-1-(4-fluorophenyl)ethanol), (S)-2-(4-Fluorophenyl)oxirane, or (1R,2S)-2-(4-fluorophenyl)cyclopropanamine hydrochloride.

In certain embodiments, the ketoreductase enzyme yields (S) enantiomeric excess of ≥95%; in certain embodiments, the ketoreductase enzyme yields (S) enantiomeric excess of ≥97%; in certain embodiments, the ketoreductase enzyme yields (S) enantiomeric excess of ≥98%; in certain embodiments, the ketoreductase enzyme yields (S) enantiomeric excess of ≥99%. In any of the foregoing embodiments, the (S) enantiomer may be the (S)-halohydrin.

In certain embodiments, the ketoreductase enzyme yields a high conversion rate of starting material to desired product. In certain embodiments, the ketoreductase enzyme has good temperature and solvent tolerance.

In certain embodiments, the ketoreductase is chosen from P1-A04, P1-B02, P1-B10, P1-B12, P1-C01, P1-H08, P1-H10, P2-B02, P2-C02, P2-C11, P2-D11, P1-F07, P2-G03, and P2-H07, which yielded ≥97% conversion of the acetophenone to the halohydrin. In certain embodiments, the ketoreductase is chosen from P1-A04, P1-B02, P1-B10, P1-B12, P1-H10, P2-C11, P1-F07, P2-G03, and P2-H07, which yielded ≥97% conversion of the acetophenone to the halohydrin and (S)-halohydrin enantiomeric excess of ≥97%. In certain embodiments, the ketoreductase is chosen from P1-A04, P1-B02, P1-B12, P1-H10, P2-C11, P1-F07, P2-G03, and P2-H07, which yielded ≥97% conversion of the acetophenone to the halohydrin and (S)-halohydrin enantiomeric excess of ≥98%. %. In certain embodiments, the ketoreductase is chosen from P1-A04, P1-B12, P1-H10, P1-F07, P2-G03, and P2-H07, which yielded ≥97% conversion of the acetophenone to the halohydrin and (S)-halohydrin enantiomeric excess of ≥99%. In certain embodiments, the ketoreductase is chosen from P1-F07 and P2-G03.

In certain embodiments, the provided chiral halohydrin compound is substantially pure in the enantiomer of structural formula III. In certain embodiments, the provided chiral halohydrin compound is at least 99% pure in the enantiomer of structural formula III.

In certain embodiments, the process is carried out with whole cells that express the ketoreductase enzyme, or an extract or lysate of such cells.

In certain embodiments, the ketoreductase is isolated and/or purified.

In certain embodiments, the enantioselective reduction reaction is carried out in the presence of a cofactor for the ketoreductase and optionally a regeneration system for the cofactor.

In certain embodiments, the process is carried out at a temperature in the range of about 15° C. to about 75° C.

In certain embodiments, the process is carried out at a pH in the range of about pH 5 to pH 8.

In certain embodiments, the weight ratio of the oxo compound of structural formula II to the ketoreductase enzyme is in the range of about 10:1 to 200:1.

In certain embodiments, the process is carried out in the presence of a cofactor and optionally a cofactor regeneration system. In particular embodiments, the cofactor is NADH and/or NADPH, and in which the weight ratio of the cofactor to the ketoreductase enzyme is in the range of about 10:1 to 100:1. In particular embodiments, the cofactor regenerating system comprises glucose dehydrogenase and glucose; formate dehydrogenase and formate; or isopropanol and a secondary alcohol dehydrogenase.

In certain embodiments, the base in step b. is chosen from inorganic bases, organic base, and combinations thereof. In certain embodiments, the base in step b. is chosen from NaOH, sodium t-butoxide, KOH, Mg(OH)₂, K₂HPO₄, MgCO₃, Na₂CO₃, K₂CO₃, triethylamine, diisopropylethylamine and N-methyl morpholine. In particular embodiments, the base in step b. is sodium t-butoxide.

In certain embodiments, the Wadsworth-Emmons reagent in step c. is chosen from tert-butyl diethylphosphonoacetate, potassium P,P-dimethylphosphonoacetate, trimethyl phosphonoacetate, ethyl dimethylphosphonoacetate, methyl diethylphosphonoacetate, methyl P,P-bis(2,2,2-trifluoroethyl)phosphonoacetate, triethyl phosphonoacetate, allyl P,P-diethylphosphonoacetate, and trimethylsilyl P,P-diethylphosphonoacetate. In particular embodiments, the Wadsworth-Emmons reagent in step c. is triethyl phosphonoacetate.

In certain embodiments, the base in step c. is chosen from lithium diisopropylamide, sodium bis(trimethylsilyl)amide, potassium bis (trimethylsilyl) amide lithium tetramethylpiperidide, sodium hydride, potassium hydride, sodium tert-butoxide, and potassium tert-butoxide.

In certain embodiments, the reagent in step d. is chosen from sodium hydroxide, potassium hydroxide, hydrochloric acid, and sulfuric acid. In particular embodiments, the reagent in step d. is sodium hydroxide.

In certain embodiments, the azidization reagent in step e. is chosen from sodium azide, diphenylphosphoryl azide, tosyl azide, and trifluoromethanesulfonyl azide. In particular embodiments, the azidization reagent in step e. is diphenylphosphoryl azide.

In certain embodiments, the base in step e. is chosen from triethylamine, diisopropylethylamine and N-methyl morpholine. In particular embodiments, the base in step e. is triethylamine.

In certain embodiments, the alcohol of Formula VII in step e. is chosen from 9-fluorenylmethanol, t-butanol, and benzyl alcohol. In particular embodiments, the alcohol of Formula VII in step e. is t-butanol.

In certain embodiments, the deprotecting base or acid in step f is chosen from piperidine, morpholine, hydrochloric acid, hydrobromic acid, trifluoroacetic acid, sulfuric acid, and hydrogen gas in the presence of a metal catalyst. In certain embodiments, the metal catalyst is chosen from platinum, palladium, rhodium, ruthenium, and nickel. In particular embodiments, the reagent is hydrochloric acid.

As used herein, a compound is “enriched” in a particular stereoisomer when that stereoisomer is present in excess over any other stereoisomer present in the compound. A compound that is enriched in a particular stereoisomer will typically comprise at least about 60%, 70%, 80%, 90%, or even more, of the specified stereoisomer. The amount of enrichment of a particular stereoisomer can be confirmed using conventional analytical methods routinely used by those of skill in the art, as will be discussed in more detail, below.

In certain embodiments, the amount of undesired stereoisomers may be less than 10%, for example, less than 9%, less than 8%, less than 7%, less than 6%, less than 5%, less than 4%, less than 3%, less than 2%, less than 1% or even less than 0.9%, 0.8%, 0.7%, 0.6%, 0.5%, 0.4%, 0.2%, or 0.1%. Stereoisomerically enriched compounds that contain at least about 95% or more of the desired stereoisomer are referred to herein as “substantially pure” stereoisomers. In certain embodiments, compounds that are substantially pure in a specified stereoisomer contain greater than 96%, 97%, 98%, or 99% of the particular stereoisomer. In certain embodiments, compounds that are substantially pure in a specified stereoisomer contain greater than 99.5%, 99.6%, 99.7%, 99.8%, 99.9%, 99.91%, 99.92%, 99.93%, 99.94%, 99.95%, 99.96%, 99.97%, 99.98% or even 99.99% of the particular stereoisomer. Stereoisomerically enriched compounds that contain ˜99.99% of the desired stereoisomer are referred to herein as “pure” stereoisomers. The stereoisomeric purity of any chiral compound described herein can be determined or confirmed using conventional analytical methods known in the art.

As is known by those of skill in the art, ketoreductase-catalyzed reduction reactions typically require a cofactor. Reduction reactions catalyzed by the engineered ketoreductase enzymes described herein also typically require a cofactor, although many embodiments of the engineered ketoreductases require far less cofactor than reactions catalyzed with wild-type ketoreductase enzymes. As used herein, the term “cofactor” refers to a non-protein compound that operates in combination with a ketoreductase enzyme.

Cofactors suitable for use with the engineered ketoreductase enzymes described herein include, but are not limited to, NADP⁺ (nicotinamide adenine dinucleotide phosphate), NADPH (the reduced form of NADP⁺), NAD⁺ (nicotinamide adenine dinucleotide) and NADH (the reduced form of NAD⁺).

The term “cofactor regeneration system” refers to a set of reactants that participate in a reaction that reduces the oxidized form of the cofactor (e.g., NADP⁺ to NADPH).

Cofactors oxidized by the ketoreductase-catalyzed reduction of the halo ketone are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst, that catalyzes the reduction of the oxidized form of the cofactor by the reductant. Cofactor regeneration systems to regenerate NADH or NADPH from NAD⁺ or NADP⁺, respectively, are known in the art and may be used in the methods described herein.

Suitable exemplary cofactor regeneration systems that may be employed include, but are not limited to, glucose and glucose dehydrogenase, formate and formate dehydrogenase, glucose-6-phosphate and glucose-6-phosphate dehydrogenase, a secondary (e.g., isopropanol) alcohol and secondary alcohol dehydrogenase, phosphite and phosphite dehydrogenase, molecular hydrogen and hydrogenase, and the like. These systems may be used in combination with either NADP⁺/NADPH or NAD⁺/NADH as the cofactor. Electrochemical regeneration using hydrogenase may also be used as a cofactor regeneration system. Chemical cofactor regeneration systems comprising a metal catalyst and a reducing agent.

The terms “glucose dehydrogenase” and “GDH” are used interchangeably herein to refer to an NAD⁺ or NADP⁺-dependent enzyme that catalyzes the conversion of D-glucose and NAD⁺ or NADP⁺ to gluconic acid and NADH or NADPH, respectively.

Glucose dehydrogenases that are suitable for use in the practice of the methods described herein include both naturally occurring glucose dehydrogenases, as well as non-naturally occurring glucose dehydrogenases.

Non-naturally occurring glucose dehydrogenases may be generated using known methods, such as, for example, mutagenesis, directed evolution, and the like.

Glucose dehydrogenases employed in the ketoreductase-catalyzed reduction reactions described herein may exhibit an activity of at least about 10 μmol/min/mg and sometimes at least about 102 μmol/min/mg or about 103 μmol/min/mg, up to about 104 μmol/min/mg or higher.

The ketoreductase-catalyzed reduction reactions described herein are generally carried out in a solvent. Suitable solvents include water, organic solvents (e.g., ethyl acetate, butyl acetate, 1-octanol, heptane, octane, methyl t-butyl ether (MTBE), toluene, and the like), ionic liquids (e.g., 1-ethyl 4-methylimidazolium tetrafluoroborate, 1-butyl-3-methylimidazolium tetrafluoroborate, 1-butyl-3-methylimidazolium hexafluorophosphate, and the like). In certain embodiments, aqueous solvents, including water and aqueous co-solvent systems, are used.

Exemplary aqueous co-solvent systems have water and one or more organic solvent. In general, an organic solvent component of an aqueous co-solvent system is selected such that it does not completely inactivate the ketoreductase enzyme.

The organic solvent component of an aqueous co-solvent system may be miscible with the aqueous component, providing a single liquid phase, or may be partly miscible or immiscible with the aqueous component, providing two liquid phases. Generally, when an aqueous co-solvent system is employed, it is selected to be biphasic, with water dispersed in an organic solvent, or vice-versa. Generally, when an aqueous co-solvent system is utilized, it is desirable to select an organic solvent that can be readily separated from the aqueous phase. In general, the ratio of water to organic solvent in the co-solvent system is typically in the range of from about 90:10 to about 10:90 (v/v) organic solvent to water, and between 80:20 and 20:80 (v/v) organic solvent to water. The co-solvent system may be pre-formed prior to addition to the reaction mixture, or it may be formed in situ in the reaction vessel.

The aqueous solvent (water or aqueous co-solvent system) may be pH-buffered or unbuffered. The reduction of the haloketone to the corresponding halohydrin can be carried out at a pH of about 5 or above. Generally, the reduction is carried out at a pH of about 10 or below, usually in the range of from about 5 to about 10. In certain embodiments, the reduction is carried out at a pH of about 9 or below, usually in the range of from about 5 to about 9. In certain embodiments, the reduction is carried out at a pH of about 8 or below, often in the range of from about 5 to about 8, and usually in the range of from about 6 to about 8. The reduction may also be carried out at a pH of about 7.8 or below, or 7.5 or below. Alternatively, the reduction may be carried out a neutral pH, i.e., about 7.

During the course of the reduction reactions, the pH of the reaction mixture may change. The pH of the reaction mixture may be maintained at a desired pH or within a desired pH range by the addition of an acid or a base during the course of the reaction. Alternatively, the pH may be controlled by using an aqueous solvent that comprises a buffer. Suitable buffers to maintain desired pH ranges are known in the art and include, for example, phosphate buffer, triethanolamine buffer, and the like. Combinations of buffering and acid or base addition may also be used.

When the glucose/glucose dehydrogenase cofactor regeneration system is employed, the co-production of gluconic acid (pKa=3.6), causes the pH of the reaction mixture to drop if the resulting aqueous gluconic acid is not otherwise neutralized. The pH of the reaction mixture may be maintained at the desired level by standard buffering techniques, wherein the buffer neutralizes the gluconic acid up to the buffering capacity provided, or by the addition of a base concurrent with the course of the conversion. Combinations of buffering and base addition may also be used. Suitable buffers to maintain desired pH ranges are described above. Suitable bases for neutralization of gluconic acid are organic bases, for example amines, alkoxides and the like, and inorganic bases, for example, hydroxide salts (e.g., NaOH), carbonate salts (e.g., K₂CO₃), bicarbonate salts (e.g., NaHCO₃), basic phosphate salts (e.g., K₂HPO₄, Na₃PO₄), and the like. The addition of a base concurrent with the course of the conversion may be done manually while monitoring the reaction mixture pH or, more conveniently, by using an automatic titrator as a pH stat. A combination of partial buffering capacity and base addition can also be used for process control.

In such reduction reactions when the pH is maintained by buffering or by addition of a base over the course of the conversion, an aqueous gluconate salt rather than aqueous gluconic acid is the product of the overall process.

When base addition is employed to neutralize the gluconic acid released during the ketoreductase-catalyzed reduction reaction, the progress of the conversion may be monitored by the amount of base added to maintain the pH. Typically, bases added to unbuffered or partially buffered reaction mixtures over the course of the reduction are added in aqueous solutions.

In certain embodiments, when the process is carried out using whole cells of the host organism, the whole cell may natively provide the cofactor. Alternatively or in combination, the cell may natively or recombinantly provide the glucose dehydrogenase.

The terms “formate dehydrogenase” and “FDH” are used interchangeably herein to refer to an NAD⁺ or NADP⁺-dependent enzyme that catalyzes the conversion of formate and NAD⁺ or NADP⁺ to carbon dioxide and NADH or NADPH, respectively. Formate dehydrogenases that are suitable for use as cofactor regenerating systems in the ketoreductase-catalyzed reduction reactions described herein include both naturally occurring formate dehydrogenases, as well as non-naturally occurring formate dehydrogenases. Formate dehydrogenases employed in the methods described herein, whether naturally occurring or non-naturally occurring, may exhibit an activity of at least about 1 μmol/min/mg, sometimes at least about 10 μmol/min/mg, or at least about 10² μmol/min/mg, up to about 10³ μmol/min/mg or higher.

As used herein, the term “formate” refers to formate anion (HCO₂⁻), formic acid (HCO₂H), and mixtures thereof. Formate may be provided in the form of a salt, typically an alkali or ammonium salt (for example, HCO₂Na, KHCO₂NH₄, and the like), in the form of formic acid, typically aqueous formic acid, or mixtures thereof. Formic acid is a weak acid. In aqueous solutions within several pH units of its pKa (pKa=3.7 in water) formate is present as both HCO₂⁻ and HCO₂H in equilibrium concentrations. At pH values above about pH 4, formate is predominantly present as HCO₂⁻. When formate is provided as formic acid, the reaction mixture is typically buffered or made less acidic by adding a base to provide the desired pH, typically of about pH 5 or above. Suitable bases for neutralization of formic acid include, but are not limited to, organic bases, for example amines, alkoxides and the like, and inorganic bases, for example, hydroxide salts (e.g., NaOH), carbonate salts (e.g., K₂CO₃), bicarbonate salts (e.g., NaHCO₃), basic phosphate salts (e.g., K₂HPO₄, Na₃PO₄), and the like.

When formate and formate dehydrogenase are employed as the cofactor regeneration system, the haloketone ester is reduced by the ketoreductase and NADH or NADPH, the resulting NAD⁺ or NADP⁺ is reduced by the coupled oxidation of formate to carbon dioxide by the formate dehydrogenase

The terms “secondary alcohol dehydrogenase” and “sADH” are used interchangeably herein to refer to an NAD⁺ or NADP⁺-dependent enzyme that catalyzes the conversion of a secondary alcohol and NAD⁺ or NADP⁺ to a ketone and NADH or NADPH, respectively.

Secondary alcohol dehydrogenases that are suitable for use as cofactor regenerating systems in the ketoreductase-catalyzed reduction reactions described herein include both naturally occurring secondary alcohol dehydrogenases, as well as non-naturally occurring secondary alcohol dehydrogenases. Naturally occurring secondary alcohol dehydrogenases include known alcohol dehydrogenases from, Thermoanaerobium brockii, Rhodococcus erythropolis, Lactobacillus kefiri, and Lactobacillus brevis, and non-naturally occurring secondary alcohol dehydrogenases include engineered alcohol dehydrogenases derived therefrom. Secondary alcohol dehydrogenases employed in the methods described herein, whether naturally occurring or non-naturally occurring, may exhibit an activity of at least about 1 μmol/min/mg, sometimes at least about 10 μmol/min/mg, or at least about 102 μmol/min/mg, up to about 103 μmol/min/mg or higher.

Suitable secondary alcohols include lower secondary alkanols and aryl-alkyl carbinols. Examples of lower secondary alcohols include isopropanol, 2-butanol, 3-methyl-2-butanol, 2-pentanol, 3-pentanol, 3,3-dimethyl-2-butanol, and the like. In one embodiment the secondary alcohol is isopropanol. Suitable aryl-alkyl carbinols include unsubstituted and substituted 1-arylethanols.

When a secondary alcohol and secondary alcohol dehydrogenase are employed as the cofactor regeneration system, as the haloketone is reduced by the engineered ketoreductase and NADH or NADPH, the resulting NAD⁺ or NADP⁺ is reduced by the coupled oxidation of the secondary alcohol to the ketone by the secondary alcohol dehydrogenase.

Some engineered ketoreductases also have activity to dehydrogenate a secondary alcohol reductant. In certain embodiments using secondary alcohol as reductant, the engineered ketoreductase and the secondary alcohol dehydrogenase are the same enzyme.

In carrying out embodiments of the ketoreductase-catalyzed reduction reactions described herein employing a cofactor regeneration system, either the oxidized or reduced form of the cofactor may be provided initially. In certain embodiments, cofactor regeneration systems are not used. For reduction reactions carried out without the use of a cofactor regenerating systems, the cofactor is added to the reaction mixture in reduced form.

In carrying out the enantioselective reduction reactions described herein, the engineered ketoreductase enzyme, and any enzymes comprising the optional cofactor regeneration system, may be added to the reaction mixture in the form of the purified enzymes, whole cells transformed with gene(s) encoding the enzymes, and/or cell extracts and/or lysates of such cells. The gene(s) encoding the engineered ketoreductase enzyme and the optional cofactor regeneration enzymes can be transformed into host cells separately or together into the same host cell. For example, in certain embodiments one set of host cells can be transformed with gene(s) encoding the engineered ketoreductase enzyme and another set can be transformed with gene(s) encoding the cofactor regeneration enzymes. Both sets of transformed cells can be utilized together in the reaction mixture in the form of whole cells, or in the form of lysates or extracts derived therefrom. In other embodiments, a host cell can be transformed with gene(s) encoding both the engineered ketoreductase enzyme and the cofactor regeneration enzymes.

Whole cells transformed with gene(s) encoding the engineered ketoreductase enzyme and/or the optional cofactor regeneration enzymes, or cell extracts and/or lysates thereof, may be employed in a variety of different forms, including solid (e.g., lyophilized, spray-dried, and the like) or semisolid (e.g., a crude paste).

The cell extracts or cell lysates may be partially purified by precipitation (ammonium sulfate, polyethyleneimine, heat treatment or the like, followed by a desalting procedure prior to lyophilization (e.g., ultrafiltration, dialysis, and the like). Any of the cell preparations may be stabilized by crosslinking using known crosslinking agents, such as, for example, glutaraldehyde or immobilization to a solid phase (e.g., Eupergit C, and the like).

The solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a prechilled lyophilization chamber, followed by the application of a vacuum. After the removal of water from the samples, the temperature is typically raised to 4° C. for two hours before release of the vacuum and retrieval of the lyophilized samples.

The quantities of reactants used in the reduction reaction will generally vary depending on the quantities of halohydrin desired, and concomitantly the amount of ketoreductase substrate employed. Generally, halo ketone substrates are employed at a concentration of about 20 to 300 grams/liter using from about 50 mg to about 5 g of ketoreductase and about 10 mg to about 150 mg of cofactor. Those having ordinary skill in the art will readily understand how to vary these quantities to tailor them to the desired level of productivity and scale of production. Appropriate quantities of optional cofactor regeneration system may be readily determined by routine experimentation based on the amount of cofactor and/or ketoreductase utilized. In general, the reductant (e.g., glucose, formate, isopropanol) is utilized at levels above the equimolar level of ketoreductase substrate to achieve essentially complete or near complete conversion of the ketoreductase substrate.

The order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor regeneration system, cofactor, ketoreductase, and ketoreductase substrate may be added first to the solvent.

For improved mixing efficiency when an aqueous co-solvent system is used, the cofactor regeneration system, ketoreductase, and cofactor may be added and mixed into the aqueous phase first. The organic phase may then be added and mixed in, followed by addition of the ketoreductase substrate. Alternatively, the ketoreductase substrate may be premixed in the organic phase, prior to addition to the aqueous phase

Suitable conditions for carrying out the ketoreductase-catalyzed reduction reactions described herein include a wide variety of conditions which can be readily optimized by routine experimentation that includes, but is not limited to, contacting the engineered ketoreductase enzyme and substrate at an experimental pH and temperature and detecting product, for example, using the methods described in the Examples provided herein.

The ketoreductase catalyzed reduction is typically carried out at a temperature in the range of from about 15° C. to about 75° C. For some embodiments, the reaction is carried out at a temperature in the range of from about 20° C. to about 55° C. In still other embodiments, it is carried out at a temperature in the range of from about 20° C. to about 45° C. The reaction may also be carried out under ambient conditions.

The reduction reaction is generally allowed to proceed until essentially complete, or near complete, reduction of substrate is obtained. Reduction of substrate to product can be monitored using known methods by detecting substrate and/or product. Suitable methods include gas chromatography, HPLC, and the like. Conversion yields of the haloketone reduction product generated in the reaction mixture are generally greater than about 50%, may also be greater than about 60%, may also be greater than about 70%, may also be greater than about 80%, may also be greater than 90%, and are often greater than about 97%.

### EXAMPLES

Non-limiting examples of methods for producing stereoisomerically pure aminocyclopropanes, more specifically to methods of using engineered ketoreductase enzymes to synthesize aminocyclopropanes are provided.

Unless otherwise noted, reagents and solvents were used as received from commercial suppliers. Deionized water was produced in house. Proton nuclear magnetic resonance spectra were obtained on a Bruker AVANCE 300 spectrometer at 300 MHz or Bruker AVANCE 500 spectrometer at 500 MHz. Spectra are given in ppm (d) and coupling constants, J values, are reported in Hertz. Tetramethylsilane was used as an internal standard.

Thin-layer chromatography (TLC) was performed using Analtech silica-gel plates and visualized by ultraviolet (UV) light or iodine.

### Example 1

### Ketoreductase (KRED) Selection

A KRED screen (KRED screening kit, Codexis Inc.) was conducted in 4 mL transparent glass vials in a total reaction volume of 1 mL. To about 1 mg of lyophilized enzyme powder in each vial, 0.8 mL of setup solution, consisting of 125 mM potassium phosphate, 1.25 mM magnesium sulfate, 1 mM NADP+ at pH 7.0, was added. 130 mg of 2-chloro-4′-fluoroacetophenone was dissolved in 2.47 mL of isopropyl alcohol and 0.13 mL of acetonitrile to give a clear solution. 0.2 mL of the substrate solution containing ˜10 mg of ketone was added to each vial and mixed. The reaction vials were incubated at 30° C. for 16 h with shaking (˜ 220 rpm).

Work up and analysis: After 16 h, 3 mL of ethyl acetate was added to each of the vials and mixed. The ethyl acetate layer was separated, washed with brine and dried over anhydrous sodium sulfate. Solvent was removed under nitrogen and the sample reconstituted with 100% ethanol. The reconstituted sample was analyzed by the chiral HPLC method shown below.

Chiral HPLC Method:

Column: Chiralcel OJ-H, 150 mm×4.6 mm, 5 μm particles

Temperature: ambient; Flow Rate: 1.0 mL/min

Gradient: 10% Ethanol (reagent alcohol) in heptane with 1% diethylamine

Time: 20 min; Detection: 264 nm

Results are shown below in Tables 1-4, in which “EE” means enantiomeric excess and “successful” KRED reactions are those which yielded ≥97% conversion.

Ketoreductase (KRED) Mediated Reduction of 2-Chloro-3′-Hydroxyacetophenone:

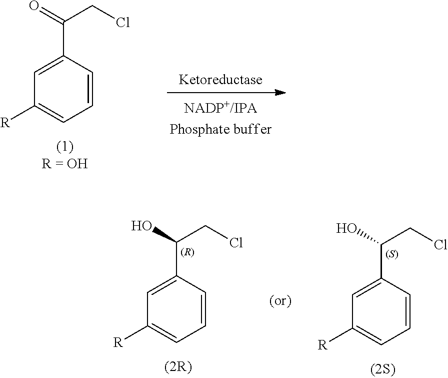

TABLE 1
Results from chiral HPLC analysis of successful KRED reactions

| | (% AUC, 220 nm) | | |
|---|---|---|---|
| | | Major isomer | |
| Enzyme | Conversion | formed | ee |
| KRED-P1-A04 | >97% | S | >99% |
| KRED-P1-B10 | >97% | S | >99% |
| KRED-P1-B12 | >97% | S | >99% |
| KRED-P1-C01 | >97% | R | 1.4% |
| KRED-P1-H08 | >97% | R | 63% |
| KRED-P2-B02 | >97% | R | >99% |
| KRED-P2-C02 | >97% | R | >99% |
| KRED-P2-C11 | >97% | S | 23.7% |
| KRED-P2-D11 | >97% | R | 37.3% |
| KRED-P2-G03 | >97% | S | 53.3% |
| KRED-P2-H07 | >97% | S | >99% |

Ketoreductase Mediated Reduction of TBS-Protected 2-Chloro-3′-Hydroxyacetophenone:

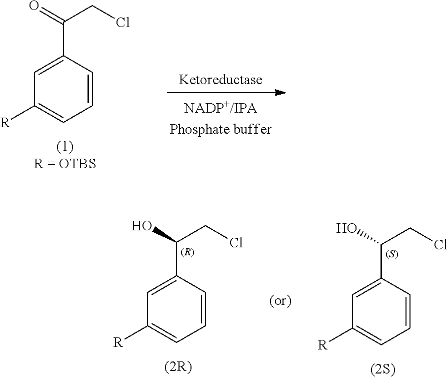

TABLE 2
Results from the KRED screen of TBS-protected
2-chloro-3′-hydroxyacetophenone

| | Conversion |
|---|---|
| Enzyme | (% AUC, 220 nm) |
| KRED-P1-A04 | 2.3 |
| KRED-P1-B02 | 70 |
| KRED-P1-B05 | 34 |
| KRED-P1-B10 | 1 |
| KRED-P1-B12 | 5 |
| KRED-P1-C01 | 78 |
| KRED-P1-H08 | 12.5 |
| KRED-P1-H10 | 6 |
| KRED-P2-B02 | 73 |
| KRED-P2-C02 | 26 |
| KRED-P2-C11 | 15.5 |
| KRED-P2-D03 | 28 |
| KRED-P2-D11 | 77 |
| KRED-P2-D12 | 1.5 |
| KRED-P2-G03 | 27 |
| KRED-P2-H07 | 0 |
| KRED-P3-B03 | 0 |
| KRED-P3-G09 | 0 |
| KRED-P3-H12 | 4.5 |

Ketoreductase (KRED) Mediated Reduction of 2-Chloro-4′-Fluoroacetophenone:

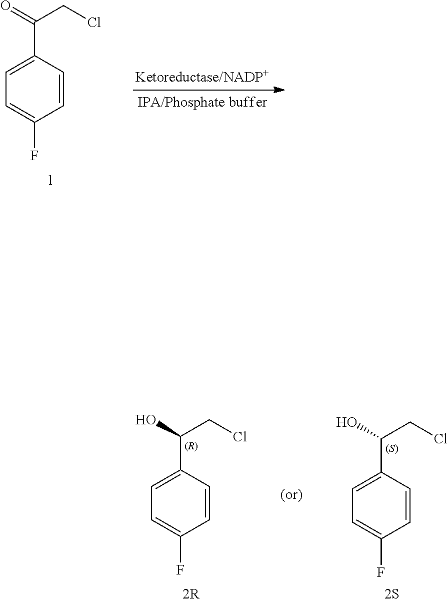

TABLE 3
Results from the KRED screen of 2-chloro-4′-fluoroacetophenone

| | Conversion |
|---|---|
| Enzyme | (% AUC, 220 nm) |
| KRED-P1-A04 | >97% |
| KRED-P1-B02 | >97% |
| KRED-P1-B05 | 37% |
| KRED-P1-B10 | >97% |
| KRED-P1-B12 | >97% |
| KRED-P1-C01 | >97% |
| KRED-P1-H08 | >97% |
| KRED-P1-H10 | >97% |
| KRED-P2-B02 | >97% |
| KRED-P2-C02 | >97% |
| KRED-P2-C11 | >97% |
| KRED-P2-D03 | 30% |
| KRED-P2-D11 | >97% |
| KRED-P2-D12 | 47% |
| KRED-P2-G03 | >97% |
| KRED-P2-H07 | >97% |
| KRED-P3-B03 | No Conversion |
| KRED-P3-G09 | 2.5% |
| KRED-P3-H12 | 5% |

Ketoreductase (KRED) Mediated Reduction of 2-Chloro-4′-Fluoroacetophenone:

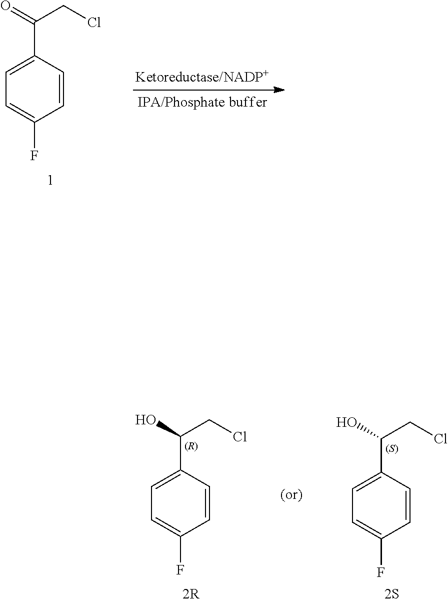

TABLE 4
Results from chiral HPLC analysis of successful KRED reactions

| KRED ID | Major isomer formed | ee |
|---|---|---|
| P1-A04 | S | >99% |
| P1-B02 | S | 97.9 |
| P1-B10 | S | 96.8 |
| P1-B12 | S | 98.6 |
| P1-C01 | S | 69.0 |
| P1-H08 | R | 91.8 |
| P1-H10 | S | 99.6 |
| P2-B02 | R | 9.0 |
| P2-C02 | R | 72.8 |
| P2-C11 | S | 98.4 |
| P2-D11 | S | 68.8 |
| P2-G03 | S | 99.6 |
| P2-H07 | S | >99% |

Scale-Up Optimization

In an effort to assess and identify optimum scale up conditions for ketoreductase (KRED) mediated stereoselective reduction of 2-chloro-4′-fluoroacetophenone to the S-halohydrin intermediate as a key step to the chiral epoxide, KRED P2-G03 was compared to an additional ketoreductase, KRED P1-F07. Reaction time course was set up using the following conditions: 150 g/L ketone, 0.5 g/L KRED, 0.1 g/L NADP, 20% v/v IPA in 0.1 M TEA buffer, pH 7+1 mM MgSO₄, at a temperature of 35° C.

P1-F07 was identified as best enzyme for scale up of ketone reduction to the desired k-halohydrin, showing slightly improved enantioselectivity and rate, as well as similar availability to P2-G03. P1-F07 was designed for better temperature and solvent tolerance: after 24 h, P1-F07 achieved enantiomeric excess of >99%, as opposed to P2-G03 which achieved enantiomeric excess of >98%, with a 99% conversion to the desired S-halohydrin. Conversion to the halohydrin was significantly higher at 4 and 6 h time period for P1-F07 when compared to P2-G03 at 35° C.

### Example 2

### Synthesis of (1R,2S)-2-(4-fluorophenyl)cyclopropanamine hydrochloride

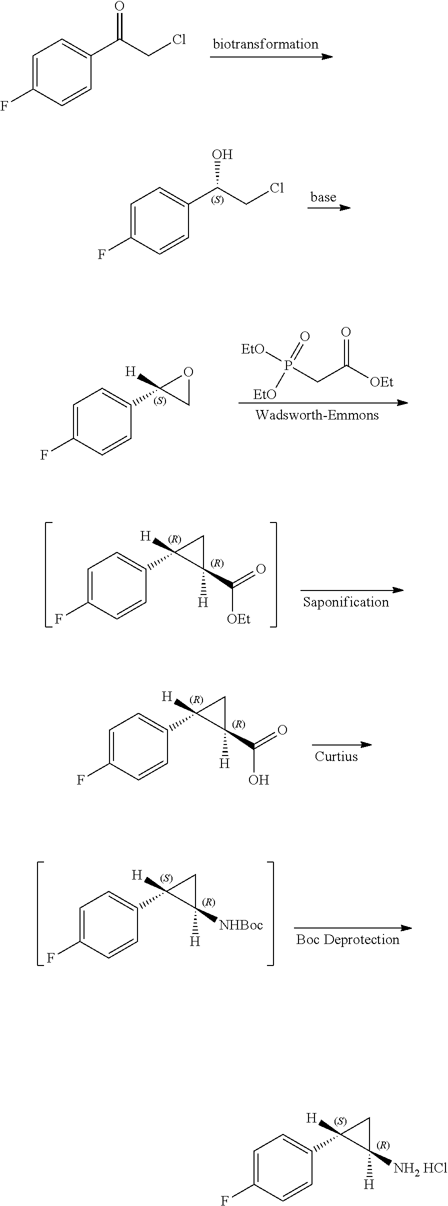

### Step 1: Synthesis of (S)-2-Chloro-1-(4-fluorophenyl)ethanol

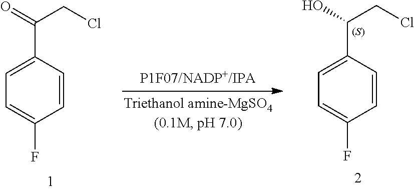

Preparation of 10 L of 0.1 M triethanolamine HCl (TEA) buffer containing 1 mM MgSO₄ (pH 7.0): Triethanolamine HCl salt (186 g, 1 mol) was dissolved in 8 L of deionized water at ambient temperature with mixing. The pH was found to be 5.3. The pH of the solution was adjusted to 7.0 using triethanolamine (free base). The solution was made up to 10 L using deionized water. 1.2 g of magnesium sulfate was added to the buffer solution and mixed. The pH of the solution was measured after the addition of MgSO₄ and found to be stable at pH 7.0.

Preparation of Buffer-Enzyme-NADP⁺ solution: Ketoreductase enzyme (3.33 g, P1F07/CDX023) from Codexis Inc., Lot #D12109; 0.5 g/L final concentration) and NADP⁺ (666 mg, 0.87 mmol) were dissolved in 1.33 L of triethanolamine HCl buffer with gentle mixing at ambient temperature for 20 minutes.

Preparation of chloroketone-IPA solution: 2-Chloro-4′-fluoroacetophenone (1 kg, 5.79 mol) was charged to a 4 L reactor (3 L working volume) equipped with overhead stirrer, addition port, and temperature probe. Isopropyl alcohol (1.33 L, 17.4 mol, 3 eq, 20% v/v final concentration) was charged with stirring and the initially formed suspension warmed to 50° C. until a clear solution was obtained.

Ketoreductase reaction procedure: 4 L of triethanolamine buffer was charged to a 12 L reactor (10 L working volume) equipped with overhead stirrer, addition port, temperature probe, nitrogen inlet, and level sensor controller. 1.33 L of buffer-enzyme-NADP⁺ solution prepared earlier was charged to the reactor. The agitation rate was set to 185 rpm, temperature set at 35° C. and nitrogen flow to 10 L/min. After the buffer-enzyme-NADP⁺ solution warmed up to 35° C. (˜20 min), the warm chloroketone-IPA solution was quickly charged to the reactor resulting in a turbid suspension. The level sensor controller was setup to replenish isopropyl alcohol/buffer that is lost due to evaporation during the course of the reaction. One arm of the level sensor controller was placed at the surface, just in contact with the suspension while the other arm was inserted deep in to the suspension. The level sensor controller was connected to a peristaltic pump in order to automatically deliver a 1:1 ratio of buffer-IP A (pre-mixed) through the addition port. The controller was setup to add the buffer-IPA mix when the level of the suspension in the reactor fell below the arm of the sensor.

Using this automated addition system, the total volume of buffer-IP A (1:1) added to the reaction over 24 h was ˜1 L.

Reaction monitoring and HPLC analysis: At periodic time intervals (4 h, 10 h and 23 h), a small aliquot of the reaction (˜2 mL) was withdrawn and diluted to 10 mL using acetonitrile (HPLC grade). The resulting suspension was centrifuged (microcentrifuge, 14,000 rpm, 5 min) and the supernatant analyzed by reversed-phase HPLC after appropriate dilution using acetonitrile (usually 40×). Details of the RP-HPLC method are shown in HPLC Method-1, below.

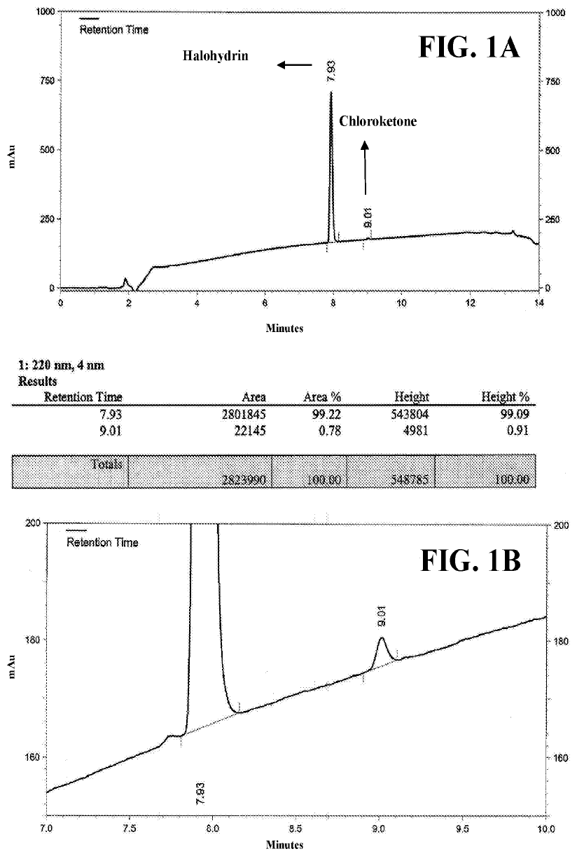

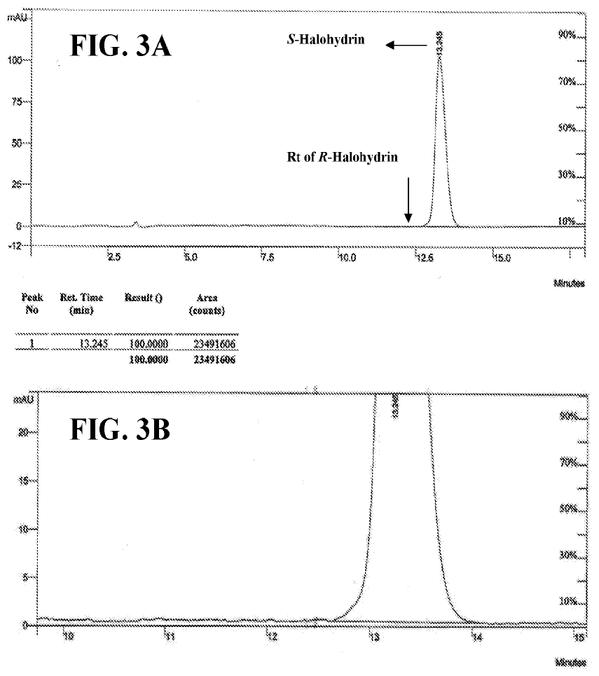

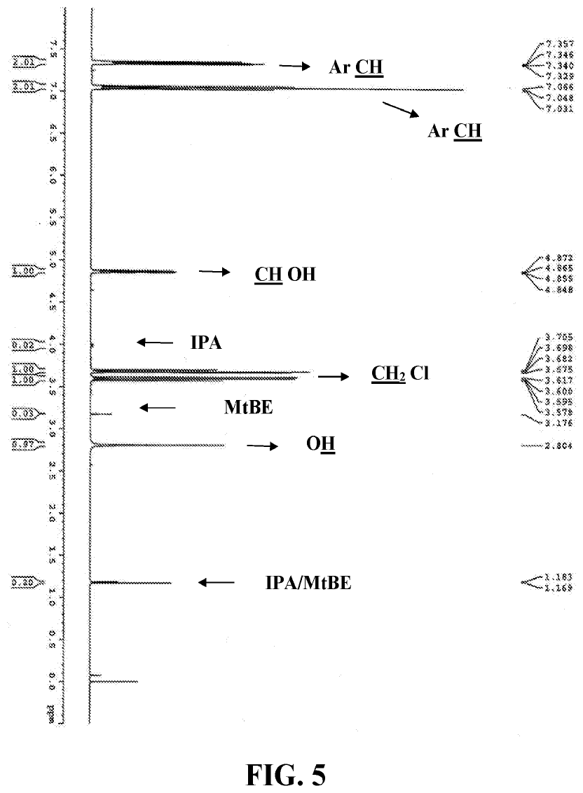

Reaction workup: After the completion of the reaction (24 h), the suspension was drained into a 20 L separatory funnel fitted with an overhead stirrer. The reaction vessel was rinsed with 7 L of MTBE and the MTBE layer drained into the same 20 L separatory funnel. After thorough mixing the layers were allowed to separate. The aqueous layer was extracted again with 7 L of MTBE. The combined MTBE layers were washed with brine, dried over anhydrous sodium sulfate, filtered and concentrated to afford a pale yellow oil. The oil was left under high vacuum for ˜48 hours to remove any residual isopropyl alcohol and MTBE. After high vacuum drying the resulting two lots of target S-halohydrin, Lot 1 and Lot 2, were found to weigh 500.5 g and 506.5 g respectively (˜98.5% isolated yield). The two lots were analyzed by ¹H NMR, RP-HPLC and chiral HPLC. The HPLC results showed >99.2% chemical (% AUC 220 nm, FIGS. 1 & 2) and >99.2% chiral purity for the isolated S-halohydrin (% AUC, 264 nm, FIGS. 3 & 4). Details of the chiral HPLC method are shown in HPLC Method-2. ¹H NMR (CDCl₃, 500 MHz) showed an estimated ˜1% of total residual solvents (IPA and MTBE) in the target halohydrin (FIGS. 5 & 6). ¹H NMR (500 Hz, CDCl₃): Lot No. 1, 7.36-7.33 (m, 2H), 7.07-7.03 (m, 2H), 4.86 (dd, J=8.5, 3.5 Hz, 1H), 3.69 (dd, J=11.5, 3.5 Hz, 1H), 3.60 (dd, J=11.0, 8.5 Hz, 1H), 2.80 (s, 1H); Lot No. 2, 7.37-7.34 (m, 2H), 7.07-7.04 (m, 2H), 4.87 (dd, J=8.5, 3.5 Hz, 1H), 3.70 (dd, J=11.0, 3.5 Hz, 1H), 3.61 (dd, J=11.0, 8.5 Hz, 1H), 2.71 (s, 1H).

### Step 2: Synthesis of (S)-2-(4-Fluorophenyl)oxirane

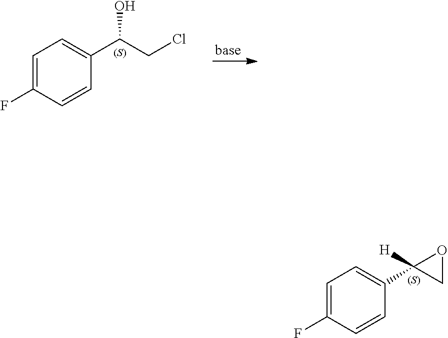

Method 1: tBuOK in THF solution. (S)-2-Chloro-1-(4-fluorophenyl)ethanol (91.7 g, 525 mmol) was charged to a 2-L, three-neck, round-bottom flask equipped with overhead stirrer, additional funnel, temperature probe, and nitrogen inlet. THF (220 mL, 2.4 vol, 99.9% purity) was added. The solution was cooled to 0-10° C. with a water-ice bath. KOtBu (1 M in THF, 657 mL, 657 mmol, 7.1 vol, 1.25 equiv) was added over 20 min slowly, keeping the internal temperature below 15° C. The reaction was stirred between 0-15° C. for 4 h (Note: HPLC indicated >99% conversion after 1 h). Water (deionized, 275 mL, 3 vol) was added to quench the reaction while keeping the internal temperature below 20° C. The ice-water bath was removed and the reaction mixture was stirred until a clear solution formed. The batch was transferred to a round-bottom flask and concentrated under reduced pressure with a rotovap below 40° C. to remove most of THF. The mixture was extracted with dichloromethane (500 mL, 400 mL, 99.96% purity), dried over anhydrous Na₂SO₄, filtered, and concentrated carefully under reduced pressure with a rotovap below 30° C. (Note: the oxirane is volatile) to give (S)-2-(4-fluorophenyl)oxirane as a light brown oil (69.2 g, 93.1%, >99.7% purity (AUC) by HPLC analysis, tR=8.53 min); ¹H NMR analysis was consistent with the assigned structure.

Method 2: NaOH in mixed DCM/water. (S)-2-Chloro-1-(4-fluorophenyl)ethanol (104 g, 600 mmol) was charged to a 2-L, three-neck, round-bottom flask equipped with overhead stirrer, additional funnel, and temperature probe. DCM (600 mL, 6 vol, 99.96% purity) was added and the solution was stirred at ambient temperature. 2 M NaOH solution [prepared by dissolving 36 g of solid NaOH (97% purity) in deionized water to 450 mL, 900 mmol, 3 vol, 1.5 equiv] was added. The reaction was stirred at ambient temperature for 23 h and then transferred to a 2-L, separatory funnel. The DCM layer was separated and the aqueous phase was extracted with DCM (100 mL). The combined organic extracts were dried over anhydrous sodium sulfate (Na₂SO₄, 20 g) for 3 h, filtered, and concentrated carefully under reduced pressure with a rotovap below 30° C. (Note: the oxirane is volatile) to about 90 g. The product was continued drying in high vacuum at ambient temperature to 82.0 g: (light yellow oil, 99.7% yield; >99.5% purity (AUC) by HPLC analysis, tR=8.52 min); ¹H NMR analysis was consistent with the assigned structure; ¹H NMR (500 Hz, CDCl3): 7.27-7.23 (m, 2H), 7.06-7.01 (m, 2H), 3.85 (dd, 7=4.0, 2.5 Hz, 1H), 3.14 (dd, 7=5.5, 4.0 Hz, 1H), 2.77 (dd, 7=5.0, 2.5 Hz, 1H).

### Step 3: Synthesis of (1R,2R)-2-(4-Fluorophenyl)cyclopropanecarboxylic acid

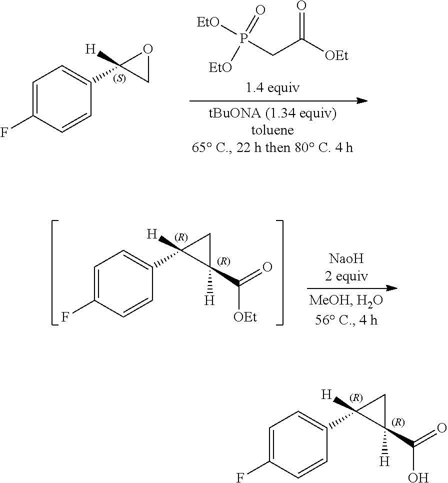

Tert-BuONa (53.4 g, 556 mmol, 1.34 equiv, 98.9% purity) was charged to a 1-L, four-neck, round-bottom flask equipped with overhead stirrer, addition funnels, temperature probe, and nitrogen inlet. Toluene (anhydrous, 230 mL, 4 vol to the epoxide, 99.8% purity, 99.96% purity) and THF (anhydrous, 57 mL, 1 vol to the epoxide, 99.9% purity) were added. After cooling below 15° C. with an ice-water bath, ethyl 2-(diethoxyphosphoryl)acetate (130 g (115 mL), 581 mmol, 1.4 equiv, 98.6% purity) was added slowly while keeping the internal temperature below 30° C. After addition, the ice-water bath was removed. The reaction mixture was stirred at ambient temperature for 1 h to afford a clear solution. Then, the reaction mixture was heated with heating mantle to 65° C. in 15 min and epoxide (S)-2-(4-fluorophenyl)oxirane (57.4 g, 41.5 mmol) was added slowly over 20 min while the reaction being heated (note: exothermal reaction). The internal temperature reached to 70.5° C. and returned back to 65° C.). After heating at 65° C. for 16 h, the reaction mixture was heated at 80° C. for additional 4 h. The reaction mixture was cooled to 45° C., quenched by addition of water (50 mL, 1.4 vol), and concentrated under reduced pressure at that temperature to remove most of the toluene to give a thick solution. MeOH (170 mL, 3 vol, 99.99% purity) and NaOH solution (prepared by dissolving 33.2 g of solid NaOH (97% purity) in deionized water to 170 mL, 830 mmol, 3 vol) were added. The solution was heated at 65° C. for 4 h and stirred at ambient temperature for 15 h. The mixture was concentrated to a slurry under reduced pressure by heating at 30-45° C. After removing about 150 mL of MeOH, water (deionized, 300 mL, 5 vol) was added, and the resulted solution was transferred to a 1-L, addition funnel. 6 N HCl (prepared by diluting concentrated HCl (105 mL, 37.% w/w) in deionized water to 210 mL, 1.3 mole, 3 vol) was charged to a separate 2-L, three-neck, round-bottom flask equipped with overhead stirrer, additional funnels, and temperature probe. The acid (50 mg) was added at ambient temperature as seeds for crystallization. After cooling to 0-5° C. with an ice-water bath, the above reaction mixture was added slowly under stirring while keeping the internal temperature below 20° C. Off-white solid was formed and the mixture was continued stirring at ambient temperature for 5 h. (Note: if no solid formed, concentrate the mixture and neutralize back to pH >8; repeat the above procedure.) Off-white solid was filtered, washed with water, air-dried for seven days then dried in high vacuum at 40° C. for 10 h to give (1R,2R)-2-(4-nuorophenyl)cyclopropanecarboxylic acid as a light yellow solid: 72.7 g; 96.9% yield; KF=0.1%; 95.6% purity (AUC) by HPLC analysis, tR=7.49 min; ¹H NMR analysis was consistent with the assigned structure; ¹H NMR (500 Hz, CDCl3): 7.10-7.06 (m, 2H), 7.10-7.06 (m, 2H), 2.61-2.57 (m, 1H), 1.87-1.83 (m, 1H), 1.67-1.63 (m, 1H), 1.38-1.34 (m, 1H).

### Steps 4-5: Synthesis of (1R,2S)-2-(4-Fluorophenyl)cyclopropanamine Hydrochloride

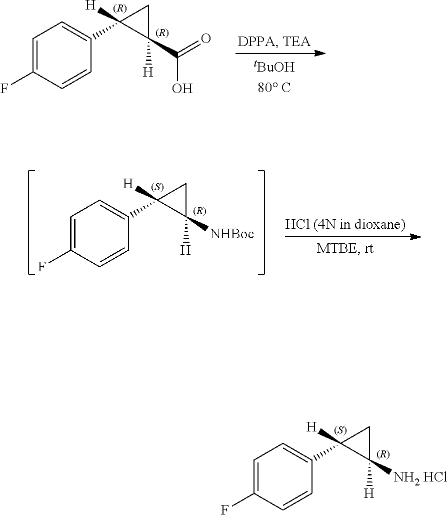

### Step 4: Curtius Rearrangement

(1R,2R)-2-(4-fluorophenyl)cyclopropanecarboxylic acid (68.0 g, 378 mmol) was charged to a 2-L, four-neck, round-bottom flask equipped with overhead stirrer, additional funnel, temperature probe, reflux condenser, and nitrogen inlet. tBuOH (anhydrous, 500 mL, 7.4 vol, 99.7% purity) was added under stirring. After forming a clear solution (note: the mixture can be heated up to 30° C. to dissolve the acid faster), DPPA (89.6 mL, 416 mmol, 1.1 equiv, 98.2% purity) was added at ambient temperature. Triethylamine (TEA) (79.0 mL g, 567 mmol, 1.5 equiv, 99.99% purity) was then added dropwise at ambient temperature in 5 min. The internal temperature elevated to 37° C. in 30 min, then lowered back to ambient temperature. The reaction mixture was heated at 80° C. (note: exothermal reaction; the reaction occurred quickly in first hour; in case of solid tBuOH accumulation in reflux condenser, stop cooling water; the reaction will be smooth after the first hour) for 20 h.

The reaction mixture was concentrated under reduced pressure to a thick solution (about 250 mL of tBuOH was removed) at 40-45° C., diluted with MTBE (800 mL, 12 vol, 99.96% purity), and washed with aqueous solutions 2 N HCl (2×100 mL, prepared by diluting concentrated HCl (33.6 mL, 37% w/w) in deionized water to 200 mL), 2 N NaOH (2×100 mL, prepared by dissolving 16 g of solid NaOH 97% purity in deionized water to 200 mL) and water (100 mL, deionized). The organic phase was transferred to 2-L, four-neck, round-bottom flask equipped with overhead stirrer, additional funnel, temperature probe, and nitrogen inlet. The mixture was concentrated under reduced pressure at 40° C. to about 4 vol and used in next step.

### Step 5: Deprotection

HCl (4 N in dioxane, 378 mL, 4.0 equiv, 4 vol) was added to above MTBE suspension at ambient temperature in 20 min and a brown solution formed. The internal temperature elevated to 37° C., then lowered back to ambient temperature. After stirring at ambient temperature for 18 h, no desired white needle-like solid was observed. The mixture was cooled with ice-water bath and white crystals formed. After stirring at that temperature for 2 h, the white crystals were filtered, washed with MTBE, and dried in high vacuum at 40° C. overnight to give the first crop: 14.5 g; 100% purity (AUC) by HPLC analysis, tR=7.49 min; estimated ee: >99%; 1H NMR analysis was consistent with the assigned structure; 1H NMR (300 Hz, DMSO-d6): 8.53 (br s, 3H), 7.24-7.18 (m, 2H), 7.16-7.09 (m, 2H), 2.80-2.74 (m, 1H), 2.39-2.32 (m, 1H), 1.43-1.36 (m, 1H), 1.22-1.15 (m, 1H).

The filtrate was concentrated under reduced pressure and dried in high vacuum overnight at ambient temperature. The residue was suspended in MTBE and dioxane. The mixture was stirred at ambient temperature for 2 h. The off-white solid was filtered, washed with MTBE, and dried in high vacuum over weekend at 40° C. to give second crop: 26.5 g; 96.1% purity (AUC) by HPLC analysis, tR=7.49 min; 1H NMR analysis was consistent with the assigned structure.

The brown filtrate was concentrated and the residue was agitated in dioxane (120 mL) and MTBE (30 mL) for 3 h. The white solid was filtered, washed with MTBE, and dried in high vacuum over weekend at 40° C. to give third crop: 9.1 g; >99.0% purity (AUC) by HPLC analysis, tR=7.49 min; 1H NMR analysis was consistent with the assigned structure. All three crops were combined to give (1R,2S)-2-(4-fluorophenyl)cyclopropanamine hydrochloride: 50.1 g, 70.6% yield.

### HPLC Methods:

### HPLC Method-1: RP-HPLC Method to Follow Halohydrin Formation

Sample preparation: 2 mL of reaction mixture was withdrawn and diluted to 10 mL using acetonitrile (HPLC grade). The resulting suspension was centrifuged (microcentrifuge, 14,000 rpm, 5 min). 25 μL of the supernatant was diluted to 1 mL using acetonitrile and the diluted sample analyzed by the following HPLC method.
Column: SunFire C18, 150 mm×4.6 mm, 3.5 μm particles
Temperature: ambient
Flow Rate: 1.0 mL/min
Injection volume: 5 μL
Gradient:

| Time | Water (%) | Acetonitrile (%) |
|---|---|---|
| (min) | (0.1% v/v formic acid) | (0.1% v/v formic acid) |
| 0 | 85 | 15 |
| 12 | 5 | 95 |
| 15 | 5 | 95 |
| 15.1 | 85 | 15 |
| 20 | 85 | 15 |

Detection: Photodiode array from 190 nm-370 nm (extraction at 220 nm)
Retention times observed for the ketone and halohydrin using the above method were ca. 9.0 min and ca. 7.9 min respectively.

### HPLC Method-2: Chiral HPLC Method.

Sample preparation: HPLC sample was prepared by dissolving ˜1.5 mg of the target halohydrin in 1 mL of HPLC grade ethanol.
Column: Chiralcel OJ-H, 150 mm×4.6 mm, 5 μm particles
Temperature: ambient
Flow Rate: 1.0 mL/min
Injection volume: 3-5 μL
Gradient: 10% Ethanol (reagent alcohol) and 90% heptane (v/v) with 0.1% v/v of diethylamine isocratic for 20 min
Detection: 264 nm
Retention time of (S)-halohydrin using the above method varied between 12.6 to 13.1 min and that of (R)-halohydrin varied between 11.4-11.9 min.

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A composition comprising:
(a) a compound of Formula II: 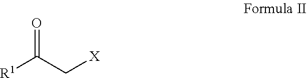
or a salt thereof; wherein:
X is chosen from Cl, Br, and I;
R¹ is chosen from aryl and heteroaryl, any of which is optionally substituted with between 1 and 3 R³ groups;
each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵; and
R⁴ and R⁵ are independently chosen from hydrogen, and lower alkyl; or R⁴ and R⁵ may be taken together to form a nitrogen-containing heterocycloalkyl or heteroaryl ring, which is optionally substituted with lower alkyl; and
(b) an engineered or isolated ketoreductase enzyme capable of stereo selectively reducing the oxo of Formula II to a hydroxyl group.

2. (canceled)

3. The composition as recited in claim 1, wherein R¹ is phenyl, which is optionally substituted with between 1 and 3 R³ groups.

4.-7. (canceled)

8. The composition as recited in claim 3, wherein R³ is halogen.

9. The composition as recited in claim 8, wherein R³ is fluorine.

10. The composition as recited in claim 1, wherein the ketoreductase enzyme converts more than about 90% of the substrate to the (S) enantiomer of the chiral halohydrin.

11. (canceled)

12. A process for preparing a chiral halohydrin compound of Formula III: 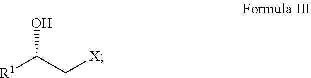
or a salt thereof; wherein:
X is chosen from Cl, Br, and I;
R¹ is chosen from aryl and heteroaryl, any of which is optionally substituted with between 1 and 3 R³ groups;
each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵; and
R⁴ and R⁵ are independently chosen from hydrogen, and lower alkyl; or R⁴ and R³ may be taken together to form a nitrogen-containing heterocycloalkyl or heteroaryl ring, which is optionally substituted with lower alkyl;
comprising the step of:
(a) enantioselectively reducing a compound of Formula II: 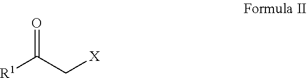
or a salt thereof; with an engineered or isolated ketoreductase enzyme capable of stereo selectively reducing the oxo to a hydroxyl group to provide the chiral halohydrin compound of Formula III:

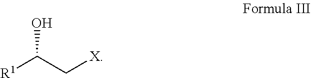

13. (canceled)

14. (canceled)

15. The process as recited in claim 12, wherein R¹ is phenyl, which is optionally substituted with between 1 and 3 R³ groups.

16.-19. (canceled)

20. The process as recited in claim 15, wherein R³ is halogen.

21. The process as recited in claim 15, wherein R³ is fluorine.

22. The process as recited in claim 12, wherein the ketoreductase enzyme converts more than about 90% of the substrate to the (S) enantiomer of the chiral halohydrin.

23. (canceled)

24. The process as recited in claim 12 in which the provided chiral halohydrin compound is substantially pure in the enantiomer of structural formula III.

25. (canceled)

26. (canceled)

27. (canceled)

28. The process as recited in claim 12, wherein the enantioselective reduction reaction is carried out in the presence of a cofactor for the ketoreductase and optionally a regeneration system for the cofactor.

29.-31. (canceled)

32. The process as recited in claim 12 in which X is chloro.

33.-35. (canceled)

36. A process for preparing a chiral cyclopropyl compound of Formula I 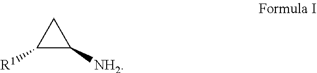
or a salt thereof; wherein:
R¹ is chosen from aryl and heteroaryl, any of which is optionally substituted with between 1 and 3 R³ groups;
R² is chosen from hydrogen and C(O)OR³;
each R³ is chosen from hydrogen, halogen, alkyl, alkenyl, alkynyl, cycloalkyl, haloalkyl, haloalkoxy, aryl, aralkyl, heterocycloalkyl, heteroaryl, heteroarylalkyl, cyano, alkoxy, amino, alkylamino, dialkylamino, C(O)R⁴, S(O)₂R⁴, NHS(O)₂R⁴, NHS(O)₂NHR⁴, NHC(O)R⁴, NHC(O)NHR⁴, C(O)NHR⁴, and C(O)NR⁴R⁵; and
each R⁴ and R⁵ are independently chosen from hydrogen, and lower alkyl; or R⁴ and R³ may be taken together to form a nitrogen-containing heterocycloalkyl or heteroaryl ring, which is optionally substituted with lower alkyl;
comprising the steps of:
(a) enantioselectively reducing a compound of Formula II: 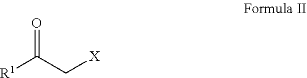
or a salt thereof; with an engineered or isolated ketoreductase enzyme capable of stereoselectively reducing the oxo to a hydroxyl group to provide a chiral halohydrin compound of Formula III: 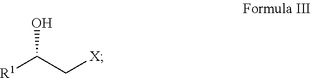
wherein X is chosen from Cl, Br, and I,
(b) treating the compound of Formula III with a base to provide the epoxide of Formula IV, or a salt thereof: 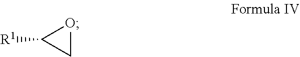
(c) treating the compound of Formula IV with a Wadsworth-Emmons reagent and a base to provide the cyclopropyl ester of Formula V, or a salt thereof: 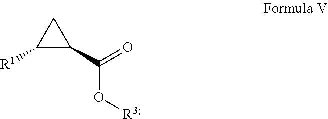
(d) treating the compound of Formula V with a reagent to provide the cyclopropyl acid of Formula VI, or a salt thereof: 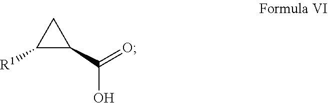
(e) treating the compound of Formula VI with azidization reagent, a base, and a alcohol of Formula VII: 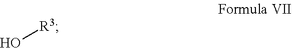
to provide the cyclopropyl carbamate of Formula VIII, or a salt thereof: 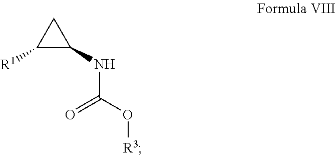
and, optionally,
(f) treating the cyclopropyl carbamate of Formula VIII with a suitable deprotecting base or acid to provide the cyclopropyl amine of Formula IX, or a salt thereof: 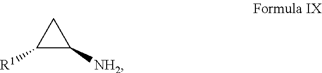
or a salt thereof.

37. (canceled)

38. (canceled)

39. The process as recited in claim 36, wherein R¹ is phenyl, which is optionally substituted with between 1 and 3 R³ groups.

40.-43. (canceled)

44. The process as recited in claim 39, wherein R³ is halogen.

45. The process as recited in claim 44, wherein R³ is fluorine.

46. The process as recited in claim 36, wherein the ketoreductase enzyme converts more than about 90% of the substrate to the (S) enantiomer of the chiral halohydrin.

47. (canceled)

48. The process as recited in claim 36 in which the provided chiral halohydrin compound is substantially pure in the enantiomer of structural formula III.

49.-51. (canceled)

52. The process as recited in claim 36, wherein the enantioselective reduction reaction is carried out in the presence of a cofactor for the ketoreductase and optionally a regeneration system for the cofactor.

53.-55. (canceled)

56. The process as recited in claim 36 in which X is chloro.

57.-61. (canceled)

62. The process as recited in claim 36, wherein the Wadsworth-Emmons reagent in step (c) is chosen from tert-butyl diethylphosphonoacetate, potassium P,P-dimethylphosphonoacetate, trimethyl phosphonoacetate, ethyl dimethylphosphonoacetate, methyl diethylphosphonoacetate, methyl P,P-bis(2,2,2-trifluoroethyl)phosphonoacetate, triethyl phosphonoacetate, allyl P,P-diethylphosphonoacetate, and trimethylsilyl P,P-diethylphosphonoacetate.

63. The process as recited in claim 36, wherein the Wadsworth-Emmons reagent in step (c) is triethyl phosphonoacetate.

64. The process as recited in claim 36, wherein the base in step (c) is chosen from lithium diisopropylamide, sodium bis(trimethylsilyl)amide, potassium bis(trimethylsilyl)amide lithium tetramethylpiperidide, sodium hydride, potassium hydride, sodium tert-butoxide, and potassium tert-butoxide.

65. (canceled)

66. The process as recited in claim 36, wherein step (c) is carried out in a solution comprising one or more solvents chosen from toluene, tetrahydrofuran, and a mixture thereof.

67. (canceled)

68. The process as recited in claim 36, wherein the reagent in step (d) is chosen from sodium hydroxide, potassium hydroxide, hydrochloric acid, and sulfuric acid.

69.-72. (canceled)

73. The process as recited in claim 36, wherein the azidization reagent in step (e) is chosen from sodium azide, diphenylphosphoryl azide, tosyl azide, and trifluoromethanesulfonyl azide.

74. The process as recited in claim 36, wherein the azidization reagent in step (e) is diphenylphosphoryl azide.

75. (canceled)

76. (canceled)

77. The process as recited in claim 36, wherein the alcohol of Formula VII in step (e) is chosen from 9-fluorenylmethanol, t-butanol, and benzyl alcohol.

78. The process as recited in claim 36, wherein the alcohol of Formula VII in step (e) is t-butanol.

79.-83. (canceled)

84. A compound prepared by the process of claim 12.

85. A compound prepared by the process of claim 36.
